(19) United States
(12) Patent Application Publication
(10) Pub. No.: US 2002/0103345 A1
(43) Pub. Date: Aug. 1, 2002

(54) Bispecific immunoglobulin-like antigen binding proteins and method of production

(76) Inventor: Zhenping Zhu, Saddle Brook, NJ (US)

Correspondence Address: KENYON & KENYON, ONE BROADWAY, NEW YORK, NY, 10004, US

(21) Appl. No.: 09/865,198

(22) Filed: May 24, 2001

Related U.S. Application Data
(60) Provisional application No. 60/206,749, filed on May 24, 2000.

(51) Int. Cl. C07K 16/42

(52) U.S. Cl. 530/388.15

(57) ABSTRACT

The present invention is directed to bispecific antigen-binding protein. These bispecific antigen-binding proteins are optimized in their avidity for antigen(s) but maintain their ability to function as a natural antibody, including the ability to activate complement mediated cytotoxicity and antibody dependent cellular toxicity. Natural IgG immunoglobulins are monospecific and bivalent, having two binding domains which are specific for the same epitope. By contrast, an IgG type immunoglobulin of the invention is bispecific and bivalent, having a binding domain on each light chain specific for one epitope and a binding domain on each heavy chain specific for a second epitope. The design of the present antigen-binding proteins provides for efficient production such that substantially all of the antigen-binding proteins produced are assembled in the desired configuration.

The subject invention claims benefit of U.S. Provisional Application 60/206,749, filed May 24, 2000, the contents of which are incorporated by reference in their entirety.

### FIELD OF THE INVENTION

The present invention is directed to production of immunoglobulin (Ig) type antigen-binding proteins. More particularly, the invention provides bispecific antigen-binding proteins which can exhibit properties of natural immunoglobulins. Natural IgG immunoglobulins are monospecific and bivalent, having two binding domains which are specific for the same antigen epitope. By contrast, an IgG type antigen-binding protein of the present invention can be bispecific and bivalent. The proteins of this invention have four antigen-binding sites, one on each of two light chains and one on each of two heavy chains. When the antigen binding sites on the light chain differ from those on the heavy chain, the protein is bispecific and bivalent. When the antigen binding sites are the same, the IgG type protein is monospecific and tetravalent. The design of the present antigen-binding proteins provides for efficient production of such molecules in a manner avoiding undesirable variable domain pairings.

### BACKGROUND OF THE INVENTION

Antibody specificity refers to selective recognition of the antibody for a particular epitope of an antigen. Natural antibodies, for example, are monospecific. Bispecific antibodies (BsAbs) are antibodies which have two different antigen-binding specificities or sites. Where an antigen-binding protein has more than one specificity, the recognized epitopes may be associated with a single antigen or with more than one antigen.

Valency refers to the number of binding sites which an antigen-binding protein has for a particular epitope. For example, a natural IgG antibody is monospecific and bivalent. Where an antigen-binding protein has specificity for more than one epitope, valency is calculated for each epitope. For example, an antigen-binding protein which has four binding sites and recognizes a single epitope is tetravalent. An antigen-binding protein with four binding sites, and specificities for two different epitopes is considered bivalent.

A natural antibody molecule is composed of two identical heavy chains and two identical light chains. Each light chain is covalently linked to a heavy chain by an interchain disulfide bond. The two heavy chains are further linked to one another by multiple disulfide bonds. FIG. 1 represents the structure of a typical IgG antibody. The individual chains fold into domains having similar sizes (110-125 amino acids) and structures, but different functions. The light chain comprises one variable domain (V_{L}) and one constant domain (C_{L}). The heavy chain comprises one variable domain (V_{H}) and, depending on the class or isotype of antibody, three or four constant domains (C_{H}1, C_{H}2, C_{H}³ and C_{H}⁴). In mice and humans, the isotypes are IgA, IgD, IgE, IgG, and IgM, with IgA and IgG further subdivided into subclasses or subtypes. The portion of an antibody consisting of V_{L} and V_{H} domains is designated “Fv” and constitutes the antigen-binding site. A single chain Fv (scFv) is an engineered protein containing a V_{L} domain and a V_{H} domain on one polypeptide chain, wherein the N terminus of one domain and the C terminus of the other domain are joined by a flexible linker. “Fab” refers to the portion of the antibody consisting of V_{L}, V_{H}, C_{L} and C_{H}1 domains.

The variable domains show considerable amino acid sequence variablity from one antibody to the next, particularly at the location of the antigen binding site. Three regions, called “hypervariable” or “complementarity-determining regions” (CDR's) are found in each of V_{L} and V_{H}. “Fc” is the designation for the portion of an antibody which comprises paired heavy chain constant domains. In an IgG antibody, for example, the Fc comprises C_{H}2 and C_{H}3 domains. The Fc of an IgA or an IgM antibody further comprises a C_{H}4 domain. The Fc is associated with Fc receptor binding, activation of complement-mediated cytotoxicity and antibody-dependent cellular-cytoxicity. For natural antibodies such as IgA and IgM, which are complexes of multiple IgG like proteins, complex formation requires Fc constant domains.

Finally, the “hinge” region separates the Fab and Fc portions of the antibody, providing for mobility of Fabs relative to each other and relative to Fc, as well as including multiple disulfide bonds for covalent linkage of the two heavy chains.

Multispecific antigen-binding proteins have been used in several small-scale clinical trials as cancer imaging and therapy agents, but broad clinical evaluation has been hampered by the lack of efficient production methods. The design of such proteins thus far has been concerned primarily with providing multispecificity. In few cases has any attention been devoted to providing other useful functions associated with natural antibody molecules.

In recent years, a variety of chemical and recombinant methods have been developed for the production of bispecific and/or multivalent antibody fragments. For review, see: Holliger, P. and Winter, G., Curr. Opin. Biotechnol. 4, 446-449 (1993); Carter, P. et al., J. Hematotherapy 4,463-470 (1995); Plückthun, A. and Pack, P., Immunotechnology 3, 83-105 (1997). Bispecificity and/or bivalency has been accomplished by fusing two scFv molecules via flexible linkers, leucine zipper motifs, C_{H}C_{L}-heterodimerization, and by association of scFv molecules to form bivalent monospecific diabodies and related structures. Multivalency has been achieved by the addition of multimerization sequences at the carboxy or amino terminus of the scFv or Fab fragments, by using for example, p53, streptavidin and helix-turn-helix motifs. For example, by dimerization via the helix-turn-helix motif of an scFv fusion protein of the form (scFv1)-hinge-helix-turn-helix-(scFv2), a tetravalent bispecific miniantibody is produced having two scFv binding sites for each of two target antigens.

Production of IgG type bispecific antibodies, which resemble IgG antibodies in that they possess a more or less complete IgG constant domain structure, has been achieved by chemical cross-linking of two different IgG molecules or by co-expression of two antibodies from the same cell. Chemical cross-linking is inefficient and can result in loss of antibody activity. Both methods result in production of significant amounts of undesired and non-functional species due to mispairing among the component heavy and light chains. Methods which have been employed to reduce or eliminate mispairing have other undesirable effects.

The production of undesired heterogeneous products has been a significant drawback to many of the methods employed so far. For example, in preparation of bispecific antibodies (BsAbs), in the absence of a method for insuring the proper association of the various domains, only a portion of the product is actually bispecific. One strategy developed to overcome unwanted pairings between two different sets of IgG heavy and light chains co-expressed in transfected cells is modification of the C_{H}3 domains of two heavy chains to reduce homodimerization between like antibody heavy chains. Merchant, A. M., et al., (1998) Nat. Biotechnology 16, 677-681. In that method, light chain mispairing was eliminated by requiring the use of identical light chains for each binding site of those bispecific antibodies.

In most work directed toward obtainining bispecific molecules, little attention has been paid to the maintenance of functional or structural aspects other than antigen specificity. For example, both complement-mediated cytotoxicity (CMC) and antibody-dependent cell-mediated cytotoxicity (ADCC), which require the presence and function of Fc region heavy chain constant domains, are lost in most bispecific antibodies. Coloma and Morrison created a homogeneous population of bivalent BsAb molecules with an Fc domain by fusing a scFv to the C-terminus of a complete heavy chain. Co-expression of the fusion with an antibody light chain resulted in the production of a homogeneous population of bivalent, bispecific molecules that bind to one antigen at one end and to a second antigen at the other end (Coloma, M. J. and Morrison, S. L. (1997) Nat. Biotechnology 15, 159-163). However, this molecule had a reduced ability to activate complement and was incapable of effecting CMC. Furthermore, the C_{H}3 domain bound to high affinity Fc receptor (FcγR1) with reduced affinity.

The present invention overcomes these disadvantages by providing antigen-binding proteins (1) which can be bispecific and bivalent, (2) in which constraints regarding selection of antigen-binding sites can be eliminated, (3) which have Fc constant domains and associated functions, (4) which are substantially homogeneous, and (5) which can be produced in mammalian or other cells without further processing.

### SUMMARY OF THE INVENTION

The present invention is directed to an antigen-binding protein comprising a complex of two first polypeptides and two second polypeptides which are stably associated in an immunoglobulin-like complex. The first polypeptide comprises an antigen-binding site located to the N terminus of an immunoglobulin light chain constant domain (C_{L} domain) capable of stable association with an immunoglobulin heavy chain first constant domain (C_{H}1 domain). The second polypeptide comprises an antigen-binding site located to the N terminus of a C_{H}1 domain followed by one or more heavy chain Fc region constant domains (C_{H} domains). The Fc C_{H} domains are capable of stable self association, i.e. each C_{H} domain can pair or bind to another copy of itself. Thus, antigen-binding proteins of the invention generally consist of four polypeptides and four antigen binding sites. In preferred embodiments, antigen-binding sites are provided by single chain Fvs although the antigen-binding site can also be provided by any sequence of amino acids capable of binding to an antigen. When the binding sites of the first and second polypeptides are different, the antigen-binding protein is bispecific. When they are the same, the antigen-binding protein is monospecific. Usually, though not necessarily, the polypeptides are covalently joined by disulfide bridges. In a preferred configuration, the antigen-binding proteins of the invention are bispecific and bivalent. That is, they bind to two different epitopes which may be carried on the same antigen or on different antigens.

In addition to providing for association of the polypeptide chains, Fe constant domains contribute other immunoglobulin functions. The functions include activation of complement mediated cytotoxicity, activation of antibody dependent cell-mediated cytotoxicity and Fe receptor binding. When antigen-binding proteins of the invention are administered for treatment or diagnostic purposes, the Fe constant domains can also contribute to serum half-life. The Fe constant domains can be from any mammalian or avian species. When antigen-binding proteins of the invention are used for treatment of humans, constant domains of human origin are preferred, although the variable domains can be non-human. In cases where human variable domains are preferred, chimeric scFvs can be used.

The antigen-binding sites can be specific for any antigen and can be obtained by any means. For example, a scFv can be obtained from a monoclonal antibody, or from a library of random combinations of and V_{L} and V_{H} domains.

In a preferred embodiment, the scFv binds specifically to human kinase insert domain-containing receptor (KDR). Particularly preferred are antigen-binding proteins that bind to the extracellular domain of KDR and block binding by its ligand vascular endothelial growth factor (VEGF) and/or neutralize VEGF induced activation of KDR. In another preferred embodiment, the scFv binds specifically to FIt-1. Also particularly preferred are antigen-binding proteins that bind to the extracellular domain of Flt-1 and block binding by one or both of its ligands VEGF and placental growth factor (P1GF) and/or neutralize VEGF inducd or P1GF induced activation of Flt-1.

Dual receptor blockade with the bifunctional antigen-binding protein can be more effective in inhibiting VEGF-stimulated angiogenesis. In a preferred embodiment, a recombinant bispecific bivalent antigen-binding protein is capable of blocking ligand binding for both Flt-1 and KDR from binding to their ligands, including VEGF and placenta growth factor (P1GF). Thus, a preferred bispecific bivalent antigen-binding protein interferes with KDR/VEGF, Flt-1/VEGF and/or Flt-1/PlGF interaction. Such an antigen-binding protein can be a stronger inhibitor of VEGF-stimulated mitogenesis of human endothelial cells, and of VEGF and PlGF-induced migration of human leukemia cells than its parent antibodies.

Antigen-binding proteins of the invention that block ligand binding of neutralize activation of KDR and/or Flt-1 are useful to reduce endothelial cell proliferation, angiogenesis and tumor growth and to inhibit VEGF- and PlGF-induced migration of human leukemia cells.

The present invention further includes methods for making antigen binding proteins whereby one or more recombinant DNA constructs encoding the first and second polypeptides of the invention are coexpressed in mammalian cells for a time and in a manner sufficient to allow expression and complexation and the antigen-binding protein is recovered.

In certain embodiments of the present invention, genes encoding scFv domains (V_{L} and V_{H}) are cloned and assembled into a bacterial vector which provides for scFv expression and screening. Nucleotide sequences encoding desired scFvs are linked, in frame, to sequences encoding desired heavy or light chain constant domains in a cloning vector designed to provide efficient expression in mammalian cells. Thus, two constructs, the first encoding a scFv and light chain constant domain and the second encoding a scFv and heavy chain constant domains, and which may be in the same or separate expression vectors, are transfected into a host cell and coexpressed.

The antigen-binding proteins of the invention which are bivalent and bispecific have a combination of desirable features. First, they are homogeneous. By design, mispairing of antibody heavy and light chains is greatly reduced or eliminated. For example, a typical bispecific antibody requires the use of two different heavy chains to provide two specificities. Four combinations are possible when the heavy chains are arranged into an IgG type molecule. Two of those consist of mispaired heavy chains such that the product is monospecific. Contrarywise, in proteins of the invention, all heavy chains are equivalent and mispairing does not occur. Because each heavy chain comprises a first complete binding site, and each light chain comprises a second different binding site, only one type of heavy chain and one type of light chain is required to provide bispecificity.

A second advantage of bispecific proteins of the invention is that in tetrameric form, they are bivalent for each binding specificity. A feature of a natural antibody which is missing from a dimeric BsAb is that the natural antibody is bivalent for the antibody binding site that it comprises. A dimeric BsAb is monovalent for each of the two binding sites that it comprises. This is significant for antibody function because bivalency allows for cooperativity of binding and a significant increase in binding avidity over a molecule comprising a single antigen-binding site.

A third advantage of proteins of the invention is that heavy chain constant domains which constitute the Fc region (e.g., C_{H}2 and C_{H}3 for an IgG molecule) of a natural antibody and which provide other antibody functions can be present. Furthermore, the multiple binding domains, along with the C_{L} and C_{H}1 domains, are separated from the Fc region such that functions provided by the Fc region are not impaired. Retained functions relate to the ability of the Fc to bind to certain accessory molecules (e.g., binding to cell surface and soluble Fc receptors, J chain association for IgA and IgM, S protein for IgA) and include activation of the complement pathway (complement mediated cytoxicity, CMC), recognition of antibody bound to target cells by several different leukocyte populations (antibody-dependent cell-mediated cytoxicity, ADCC) and opsonization (enhancement of phagocytosis). In addition, by avoiding the addition of large domains to the carboxy terminus of heavy chains, steric hindrance is avoided. This is significant for many of the above-mentioned functions, as well as for assembly of antibody molecules of higher order structure (e.g., IgA consists of four heavy chains, associated through two Fcs; IgM consists of ten heavy chains associated by five Fcs). Finally, the Fc heavy chain constant domains confer increased serum half-life.

A fourth advantage of proteins of the invention is that there is no requirement for processing in vitro to obtain the complete product. Though rearranged in an artificial manner, each of the domains has a natural character which allows expression in a biological system.

The present invention is also applicable to production of monospecific tetravalent antigen-binding proteins. In such proteins, all four binding sites have the same specificity. Furthermore, the invention provides a method of making contemplates monovalent bispecific antigen-binding proteins and bivalent monospecific antigen-binding proteins. For example, Fab type proteins can be made which comprise two different binding sites or two equivalent binding sites, the first binding site linked to a C_{L} domain and the second binding site linked to a C_{H}1 domain.

In a preferred embodiment, the first and second binding sites are each contributed by a single chain Fv (scFv). A scFv having a first binding specificity is fused to a C_{L} domain to form a scFv-C_{L} polypeptide, and a scFv having a second binding specificity is fused to C_{H} to form a scFv-C_{H} polypeptide. As referred to herein, a scFv-C_{H} polypeptide is defined as a scFv fused to any portion of an antibody heavy chain so long as there are two or more C_{H} domains with one of the domains being C_{H}1. A scFv-C_{L}-SCFV-C_{H} heterodimer is formed by natural association of the C_{L} and C_{H}1 constant domains. The presence of at least one C_{H}2, C_{H}3, or C_{H}4 constant domain allows pairing of two scFv-C_{L}-scFv-C_{H} heterodimers into an antigen-binding protein having four binding sites by natural association of a C_{H}2, C_{H}3, or C_{H}4 domain on one polypeptide with a copy of itself on another polypeptide.

The precise heavy chain constant domain structure is determined by desired functional characteristics. If it is desired that an antigen-binding protein have a particular isotype, C_{H} domains from an immunoglobulin of that isotype will be selected. For example, where the desired isotype is IgGI, the domain structure is (scFv)₂-C_{H}1-C_{H}2-C_{H}3, where the constant domains are from an IgG1 antibody.

This approach is employed to provide a homogenous population of IgG-like antigen-binding proteins having four antigen binding sites. Where each heterodimer comprises two different binding sites, the antigen-binding protein thus formed is bispecific and bivalent. Where the heterodimer comprises two equivalent binding sites, the antigen-binding protein formed is monospecific and tetravalent. In embodiments detailed herein, the antigen binding sites are comprised of antibody variable domains. However, the invention further contemplates bispecific molecules wherein one or more binding functions are contributed by structures chosen on the basis of known binding interactions with a particular protein or antigen of interest. For example, a portion of gp120 of HIV-1 may be selected on the basis of its ability to bind to CD4. Alternatively, a binding site may comprise an amino acid sequence corresponding to a hormone or cytokine selected on the basis of its ability to bind to its cognate receptor protein.

Certain antigen-binding proteins of the present invention are used for binding to antigen or to block interaction of a protein and its ligand. Other antigen-binding proteins of the present invention are used to promote interactions between immune cells and target cells. Finally, antigen-binding proteins of the invention are used to localize anti-tumor agents, target moieties, reporter molecules or detectable signal producing agents to an antigen of interest.

The present invention further provides antigen-binding proteins which bind to KDR and its analogs, or to other receptor molecules which are involved in angiogenesis or tumorigenesis.

### DESCRIPTION OF THE FIGURES

FIG. 1 is a schematic diagram of Bs(scFv)4-IgG and Bs(scFv)2-Fab molecules. In Bs(scFv)4-IgG, the V_{H} and V_{L} domains of a human IgGI molecule are replaced by two scFv antibodies of different specificity. Co-expression of the scFv-light and scFv-heavy chain fusion polypeptides in mammalian cells results in the formation of a bivalent, IgG-like bispecific molecule. In Bs(scFv)2-Fab, a stop codon is introduced at the C-terminal end of the heavy chain C_{H}1 domain, which results in the expression of a bivalent, Fab-like bispecific molecule (also see FIG. 2A).

FIG. 2 shows examples of expression constructs and purified Bs(scFv)4-IgG and Bs(scFv)2-Fab antibodies (the domains are not to scale). Panel A: Individual scFv constructs are fused at their 5′ ends to a leader sequence for secretion in mammalian cells, and at their 3′ ends to the C_{L} or C_{H}1 domains of a human IgG molecule. Panel B: SDS-PAGE analysis of protein-G purified Bs(scFv)4-IgG and Bs(scFv)2-Fab antibodies. Lanes 1-3 are run under non-reducing conditions. Lane 1, c-p1C11, a chimeric IgG1; Lane 2, Bs(scFv)4-IgG; Lane 3, Bs(scFv)2-Fab. Lanes 4-6 are run under reducing conditions. Lane 4, c-p1C11; Lane 5, Bs(scFv)4-IgG; Lane 6, Bs(scFv)2-Fab. Also shown are the positions of molecular weight standards.

FIG. 3 shows the results of ELISA assays for the bispecificity of Bs(scFv)4-IgG and Bs(scFv)2-Fab antibodies. Panel A shows binding of Bs(scFv)4-IgG, Bs(scFv)2-Fab and its parent antibodies to KDR ECD Ig domain deletion mutant-AP fusion proteins. Panel B shows cross-linking ELISA for detection of simultaneous binding by Bs(scFv)4-IgG and Bs(scFv)2-Fab to the two different epitopes that are located on separate KDR ECD Ig domain deletion mutants, KDR(Ig1-3) and KDR(Ig3-7)-AP. The BsAb are incubated in solution with KDR(Ig1-7)-AP, KDR(Ig1-3)-AP or KDR(Ig3-7)-AP, and transferred to a plate coated with untagged KDR(Ig1-3). The cross-linking complexes formed between the soluble phase antibody/KDR variant-AP complex and the immobilized KDR(Ig1-3) are detected by measuring the plate-bound AP activity. Data shown are mean ±SD of triplicate determinations.

FIG. 4 shows dose-dependent binding of Bs(scFv)4-IgG, Bs(scFv)2-Fab and its parent antibodies to immobilized full length KDR-AP (Panel A) and Flk-1-AP (Panel B). Data shown are mean ±SD of triplicate determinations.

FIG. 5 demonstrates inhibition of binding of KDR to immobilized VEGF by Bs(scFv)4-IgG and c-p1C11. Data shown are mean ±SD of triplicate determinations.

FIG. 6 demonstrates dose-dependent inhibition of VEGF-stimulated phosphorylation of KDR receptor by Bs(scFv)4-IgG and c-p 1C11. The KDR-transfected 293 cells were treated with various amounts of antibodies at RT for 15 min, followed by incubation with 20 ng/ml of VEGF (except the control group) at RT for additional 15 min. Phosphorylation of KDR is analyzed following the protocol previously described (Zhu et al. (1998) Cancer Res., 58, 3209-3214; Zhu et al. (1999) Cancer Lett. 136, 203-213).

### DETAILED DESCRIPTION OF THE INVENTION

The present invention provides antigen-binding proteins which are homogeneous and which can retain the functional characteristics of natural antibodies such as cooperativity of binding (avidity), and the ability to activate complement mediated cytotoxicity and antibody dependent cellular toxicity. In general, antigen-binding proteins of the invention have the constant domain structure of naturally-occurring antibodies, with complete antigen binding sites substituted for each antibody variable domain. Thus, in a naturally-occurring antibody, a single binding site is provided by a combination of a light chain variable domain (V_{L}) and a heavy chain variable domain (V_{H}), so that, for example, the four variable domains of an IgG type antibody provide two complete binding sites. In contrast, the IgG type antigen-binding proteins of the present invention have four complete binding sites, because a structure comprising a complete antigen binding site is substituted for each V_{L} and V_{H} variable domain of the naturally occurring antibody.

As used herein, unless otherwise indicated or clear from the context, antibody domains, regions and fragments are accorded standard definitions as are well known in the art. See, e.g., Abbas, A. K., et al., (1991) Cellular and Molecular Immunology, W. B. Saunders Company, Philadelphia, Pa.

The antigen binding site of a typical Fv contains six complementarity determining regions (CDRs) which contribute in varying degrees to the affinity of the binding site for antigen. Antigen binding sites comprised of fewer CDRs (e.g., three, four or five) are also functional and included within the scope of the invention. The extent of CDR and framework regions (FRs) is determined by comparison to a compiled database of amino acid sequences in which those regions have been defined according to variability among the seuqences. There are three heavy chain variable domain CDRs (CDRH1, CDRH2 and CDRH3) and three light chain variable domain CDRs (CDRL1, CDRL2 and CDRL3).

Avidity is a measure of the strength of binding between an immunoglobulin and its antigen. Unlike affinity, which measures the strength of binding at each binding site, avidity is related to both the affinity and the valency of an immunoglobulin molecule.

The proteins of the invention are derived from, or incorporate portions of antibodies of one or more immunoglobulin classes. Immunoglobulin classes include IgG, IgM, IgA, IgD, and IgE isotypes and, in the case of IgG and IgA, their subtypes.

The antigen-binding proteins of the invention resemble IgG type antibodies, in that they are heterotetramers comprising two light chains and two heavy chains. However, unlike IgG type antibodies, they have four antigen binding sites, and may have fewer constant domains provided at least C_{H}1 and one other C_{H} domain are present. The four antigen-binding sites may comprise two binding sites for each of two binding specificities, or four binding sites for one binding specificity.

In a preferred embodiment, a bispecific protein having this form may display avidity characteristics like those of naturally-occurring IgG type antibodies. For each binding specificity, the presence of two equivalent antigen binding sites allows for cooperativity of binding to antigen, as is the case for the naturally occurring IgG molecule. It will be apparent that by proper choice of heavy chain constant region, as well known to one of skill in the art, bispecific antibodies resembling antibodies of other classes, for example, IgA, IgM, and other types of antibodies can be produced.

The invention contemplates the linkage of binding domains of different specificity to heavy and light chain constant domains, such that upon pairing of heavy chains with light chains, different binding specificities become associated in single heterodimeric molecules. A population of such molecules is substantially homogeneous, in that practically all dimers comprise one binding domain having a first specificity and one binding domain having a second specificity. Dependence on the preferential natural pairing of heavy and light chains via association of C_{L} and C_{H}1 domains reduces or eliminates formation of dimers which comprise two binding domains having the same specificity. Likewise, preferential association of the heavy chains occurs via the Fc region to form the antigen-binding proteins of the invention.

In general, antigen binding proteins of the invention comprise complete C_{L} and C_{H}1 domains, which are covalently linked by an interchain disulfide bond. However, the invention also contemplates the use of modified C_{L} and C_{H}1 domains which may have amino acids deleted or inserted, and which, together, may or may not have an interchain disulfide bond, so long as the domains can associate in a stable complex.

By stable association, or complex, it is meant the under physiological conditions, the polypeptides of the antigen binding protein exist as a complex. For example, on a native gel under non-reducing conditions, the polypeptides migrate as a complex. It will be appreciated that not all antibody light chains effectively associate with any given heavy chain and vice versa. However, combinations of C_{L} and C_{H}1 constant domains which pair effectively are well known in the art and are preferred.

As with natural antibodies, the heavy chain-light chain heterodimers associate, via association of particular heavy chain constant domains, to form structures of higher order. For example, IgG type antibodies comprise two heavy chain—light chain heterodimers joined by covalent linkage in a tetrameric structure. Certain other antibody types comprise similar tetrameric structures which are incorporated into a higher order structure comprising, for example, two tetramers (IgA) or ten tetramers (IgM).

Like natural antibodies, bivalent bispecific antigen binding proteins of the invention rely on Fc constant domains and hinge regions for proper association of heavy chains. In general, the antigen-binding proteins of the invention comprise a hinge region and one or more Fc constant domains or portions thereof. It is usually desired to incorporate all Fc constant domains to retain all the associated functions. However, the invention further contemplates the inclusion of only certain constant domains, provided at least one such domain is present. As various Fc functions depend on different portions of the Fc, fewer C_{H} domains can be incorporated in the heavy chain if less than full functionality is desired. For example, significant activation of complement requires C_{H}2 of IgG or C_{H}3 of IgM. The invention also contemplates the use of modified hinge and Fc heavy chain domains which may have amino acids substituted, deleted, inserted or modified, so long as the heavy chains can associate in a stable complex.

The antigen binding sites of preferred antigen binding proteins consist of Fv regions of any desired specificity. The Fv is a single chain Fv (scFv) and consists of a V_{H} domain and a V_{L} domain, in either order, linked by a peptide linker, which allows the domains to associate to form a functional antigen binding site. (see, for example, U.S. Pat. No. 4,946,778, Ladner et al., (Genex); WO 88/09344, Creative Biomolecules, Inc., Uhston et al.) WO 92/01047, Cambridge Antibody Technology/McCafferty et al., describes the display of scFv fragments on the surface of soluble recombinant genetic display packages.

Peptide linkers used to produce scFvs are flexible peptides selected to assure proper three-dimensional folding and association of the V_{L} and V_{H} domains and maintenance of target molecule binding-specificity. Generally, the carboxy terminus of the V_{L} or V_{H} sequence is covalently linked by such a peptide linker to the amino terminus of a complementary V_{H} or V_{L} sequence. The linker is generally 10 to 50 amino acid residues, but any length of sufficient flexibility to allow formation of the antigen binding site is contemplated. Preferably, the linker is 10 to 30 amino acid residues. More preferably the linker is 12 to 30 amino acid residues. Most preferably is a linker of 15 to 25 amino acid residues. Example of such linker peptides include (Gly-Gly-Gly-Gly-Ser)₃.

V_{L} and V_{H} domains from any source can be incorporated into a scFv for use in the present invention. For example, V_{L} and V_{H} domains can be obtained directly from a monoclonal antibody which has the desired binding characteristics. Alternatively, V_{L} and V_{H} domains can be from libraries of V gene sequences from a mammal of choice. Elements of such libraries express random combinations of V_{L} and V_{H} domains and are screened with any desired antigen to identify those elements which have desired binding characteristics. Particularly preferred is a human V gene library. Methods for such screening are known in the art. V_{L} and V_{H} domains from a selected non-human source may be “humanized,” for example by substitution of CDR loops into human V_{L} and V_{H} domains, or modified by other means well known in the art to reduce immunogenicity when administered to a human.

In a physiological immune response, mutation and selection of expressed antibody genes leads to the production of antibodies having high affinity for their target antigen. The V_{L} and V_{H} domains expressed in a scFv can similarly be subject to in vitro mutation and screening procedures to obtain high affinity variants.

Vectors for construction and expression of scFvs are available which contain bacterial secretion signal sequences and convenient restriction cloning sites. V_{L} and V_{H} gene combinations encoding binding sites specific for a particular antigen are isolated from cDNA of B cell hybridomas. Alternatively, random combinations of V_{L} and V_{H} genes are obtained from genomic DNA and the products then screened for binding to an antigen of interest. Typically, the polymerase chain reaction (PCR) is employed for cloning, using primers which are compatible with restriction sites in the cloning vector. See, e.g., Dreher, M. L. et al. (1991) J. Immunol. Methods 139:197-205; Ward, E. S. (1993) Adv. Pharmacol. 24:1-20; Chowdhury, P. S. and Pastan, I. (1999) Nat. Biotechnol. 17:568-572.

To express scFvs with selected or random combinations of V_{L} and V_{H} domains, V genes encoding those domains are assembled into a bacterial expression vector. For example, a vector can be used which has sequences encoding a bacterial secretion signal sequence and a peptide linker and which has convenient restriction sites for insertion of V_{L} and V_{H} genes. Alternatively, it might be desired to first assemble all necessary coding sequences (e.g., secretion signal, V_{L}, V_{H} and linker peptide) into a single sequence, for example by PCR amplification using overlapping primers, followed by ligation into a plasmid or other vector. Where it is desired to provide a specific combination of V_{L} and V_{H} domains, PCR primers specific to the sequences encoding those domains are used. Where it is desired to create a diverse combinations of a large number of V_{L} and V_{H} domain, mixtures of primers are used which amplify multiple sequences.

Preferred bacterial vectors allow for expression of scFv linked to a coat protein of a filamentous phage. The phage coat protein most commonly used is the gene III protein of phage M13. The display of scFv on filamentous phage is particularly useful where it is desired to screen a large population of scFv for desired binding characteristics. Bacterial cells expressing the scFv-gIII protein fusion are infected with an M13 variant which allows for preferential packaging of vector DNA carrying the scFv-gIII fusion gene into phage particles into which the scFv-gIII coat protein fusion is incorporated. Each resulting phage particle displays a particular scFv and contains a vector which encodes the scFv. A population of such phage particles displaying a diverse collection of scFvs is then enriched for desired binding characteristics by a panning procedure. Typically, desired particles are immobilized on a solid surface coated with an antigen to which the desired phage particles can bind. The bound particles are collected and used to further infect bacterial cells. The panning procedure is repeated to further enrich for desired binding characteristics.

The vector encoding the scFv-gIII fusion may include a translational termination codon at the junction of the scFv and gIII coding regions. When expressed in a bacterial cell carrying a corresponding translation termination suppressor, the fusion protein is produced. When expressed in a bacterial cell without the corresponding suppressor, free scFv is produced.

Vascular endothelial growth factor (VEGF) is a key regulator of vasculogenesis during embryonic development and angiogenic processes during adult life such as wound healing, diabetic retinopathy, rheumatoid arthritis, psoriasis, inflammatory disorders, tumor growth and metastasis. VEGF is a strong inducer of vascular permeability, stimulator of endothelial cell migration and proliferation, and mediates its activity mainly through two tyrosine kinase receptors, VEGF receptor 1 (VEGFR-1), or fms-like tyrosine receptor 1 (Flt-1), and VEGF receptor 2 (VEGFR-2), or kinase insert domain-containing receptor (KDR, and Flk-1 in mice) Ferrara, N., Curr. Top. Microbiol. Immunol., 237, 1-30 (1999); Klagsbrum, M., et al., Cytokine Growth Factor Rev. 7, 259-270 (1996); Neufeld, G., et al. FASEB J. 13, 9-22 (1999). Numerous studies have shown that over-expression of VEGF and its receptor play an important role in tumor-associated angiogenesis, and hence in both tumor growth and metastasis.

Flt-1 and KDR have distinct functions in vascular development in embryos. Targeted deletion of genes encoding either receptor in mice is lethal to the embryo, demonstrating the physiological importance of the VEGF pathway in embryonic development. KDR-deficient mice have impaired blood island formation and lack mature endothelial cells, whereas Flt-1 null embryos fail to develop normal vasculature due to defective formation of vascular tubes, albeit with abundant endothelial cells. Shalaby, F., et al., Nature 376, 62-66 (1995); Fong, G. H., et al., Nature 376, 66-70 (1995). On the other hand, inactivation of Flt-1 signal transduction by truncation of the tyrosine kinase domain does not impair mouse embryonic angiogenesis and embryo development, suggesting that signaling through the Flt-1 receptor is not essential for vasculature development in the embryo. Hiratsuka, S., et al., Proc. Natl. Acad. Sci. USA, 95, 9349-9354 (1998). The biological responses of Flt-1 and KDR to VEGF in the adult also appear to be different. It is generally believed that KDR is the main VEGF signal transducer that results in endothelial cell proliferation, migration, differentiation, tube formation, increase of vascular permeability, and maintenance of vascular integrity. Flt-1 possesses a much weaker kinase activity, and is unable to generate a mitogenic response when stimulated by VEGF—although it binds to VEGF with an affinity that is approximately 10-fold higher than KDR. Flt-1 is also been implicated in VEGF and placenta growth factor (PIGF)-induced migration of monocytes/macrophage and production of tissue factor. Barleon, B., et al., Blood 87, 3336-3343 (1996); Clauss, M., et al., J. Biol. Chem. 271, 17629-17634 (1996).

In a preferred embodiment, an antigen binding protein of the present invention comprises a scFv that binds to KDR and blocks VEGF binding to KDR. scFv p1C11 (SEQ ID NOS: 27, 28) is produced from a mouse scFv phage display library. (Zhu et al., 1998). p1C11 blocks VEGF-KDR interaction and inhibits VEGF-stimulated receptor phosphorylation and mitogenesis of human vascular endothelial cells (HUVEC). This scFv binds both soluble KDR and cell surface-expressed KDR on, e.g., HUVEC with high affinity (K_{d}=2.1 nM).

In a second preferred embodiment, an antigen binding protein of the present invention comprises a scFv that binds to Flt-1 and blocks VEGF binding and/or PlGF binding to Flt-1. Mab 6.12 binds to soluble and cell surface-expressed Flt-1. scFv 6.12 comprises the V_{L} and V_{H} domains of mouse monoclonal antibody Mab 6.12 A hybridoma cell line producing Mab 6.12, has been deposited as ATCC number PTA-3344. The deposit was made under the provisions of the Budapest Treaty on the International Recognition of the Deposit of Microorganisms for the Purposes of Patent Procedure and the regulations thereunder (Budapest Treaty). This assures maintenance of a viable culture for 30 years from date of deposit. The organisms will be made available by ATCC under the terms of the Budapest Treaty, and subject to an agreement between Applicants and ATCC which assures unrestricted availability upon issuance of the pertinent U.S. patent. Availability of the deposited strains is not to be construed as a license to practice the invention in contravention of the rights granted under the authority of any government in accordance with its patent laws.

Antigen-binding proteins of the invention can have binding sites for any epitope, antigenic site or protein. Preferred antigen-binding proteins neutralize activation of receptor proteins. Of particular interest are VEGF receptors and other receptors which are involved in angiogenesis. VEGF receptors include KDR, Flk-1, Flt-1. Other factors implicated as possible regulators of angiogenesis in vivo include fibroblast growth factor (FGF), platelet derived growth factor (PDGF), epidermal growth factor (EGF). The corresponding receptors are fibroblast growth factor (FGF-R) and platelet derived growth factor receptor (PDGF-R), epidermal growth factor receptor (EGF-R). Also of interest are receptor tyrosine kinases involved in angiogenesis and/or oncogenesis. Such receptor tyrosine kinases include FLT4, HER2/neu, Tek and Tie2. Receptors of interest include human proteins and homologues from other mammals. Antibodies are known for the above listed receptors and are sources of scFv V_{L} and V_{H} domains for use in antigen binding proteins of the present invention. Antigen binding proteins of the invention which are specific for any of the listed receptors can be monospecific or bispecific. Certain bispecific antigen-binding proteins of the invention bind to two of the above listed receptors. In one preferred embodiment, such a bispecific antigen-binding protein binds to HER2 and EGF-R. In a second preferred embodiment, an antigen-binding protein of the invention binds to KDR and FLT-1.

Bispecific antigen-binding proteins of the invention can cross-link antigens on target cells with antigens on immune system effector cells. This can be useful, for example, for promoting immune responses directed against cells which have a particular antigens of interest on the cell surface. According to the invention, immune system effector cells include antigen specific cells such as T cells which activate cellular immune responses and nonspecific cells such as macrophages, neutrophils and natural killer (NK) cells which mediate cellular immune responses.

Antigen-binding proteins of the invention can have a binding site for any cell surface antigen of an immune system effector cell. Such cell surface antigens include, for example, cytokine and lymphokine receptors, Fc receptors, CD3, CD16, CD28, CD32 and CD64. In general, antigen binding sites are provided by scFvs which are derived from antibodies to the aforementioned antigens and which are well known in the art. Antigen-binding sites of the invention which are specific for cytokine and lymphokine receptors can also be sequences of amino acids which correspond to all or part of the natural ligand for the receptor. For example, where the cell-surface antigen is an IL-2 receptor, an antigen-binding protein of the invention can have an antigen-binding site which comprises a sequence of amino acids corresponding or IL-2. Other cytokines and lymphokines include, for example, interleukins such as interleukin-4 (IL-4) and interleukin-5 (IL-5), and colony-stimulating factors (CSFs) such as granulocyte-macrophage CSF (GM-CSF), and granulocyte CSF (G-CSF).

Preferred antigen-binding proteins of the invention are made by expressing a first polypeptide having a scFv linked to a C_{L} light chain constant domain and a second polypeptide having a scFv linked to a C_{H}1, C_{H}2 and C_{H}3 heavy chain constant domains. The DNA fragments coding for the scFvs can be cloned, e.g., into HCMV vectors designed to express either human light chains of human heavy chains in mammalian cells. (See, e.g., Bendig, et al., U.S. Pat. 5,840,299; Maeda, et al. (1991) Hum. Antibod. Hybridomas 2, 124-134). Such vectors contain the human cytomegalovirus (HCMV) promoter and enhancer for high level transcription of the light chain and heavy chain constructs. In a preferred embodiment, the light chain expression vector is pKN100 (gift of Dr. S. Tannan Jones, MRC Collaborative Center, London, England), which encodes a human kappa light chain, and the heavy chain expression vector is pG1D105 (gift of Dr. S. Tannan Jones), which encodes a human gamma-1 heavy chain. Both vectors contain HCMV promoters and enhancers, replication origins and selectable markers functional in mammalian cells and E. coli.

A selectable marker is a gene which encodes a protein necessary for the survival or growth of transformed host cells grown in a selective culture medium. Typical selectable markers encode proteins that (a) confer resistance to antibiotics or other toxins, e.g. ampicillin, neomycin, methotrexate, or tetracycline, (b) complement auxotrophic deficiencies, or (c) supply critical nutrients not available from complex media, e.g. the gene encoding D-alanine racemase for Bacilli. A particularly useful selectable marker confers resistance to methotrexate. For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity, prepared and propagated as described by Urlaub and Chasin (1980) Proc. Natl. Acad. Sci. USA 77, 4216. The transformed cells are then exposed to increased levels of methotrexate. This leads to the synthesis of multiple copies of the DHFR gene, and, concomitantly, multiple copies of other DNA comprising the expression vectors, such as the DNA encoding the antibody or antibody fragment.

Where it is desired to express a gene construct in yeast, a suitable selection gene for use in yeast is the trpl gene present in the yeast plasmid YRp7. Stinchcomb et al. (1979) Nature, 282, 39; Kingsman et al. (1979) Gene 7, 141. The trpl gene provides a selection marker for a mutant strain of yeast lacking the ability to grow in tryptophan, for example, ATCC No. 44076 or PEP4-1. Jones (1977) Genetics 85, 12. The presence of the trpl lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

Preferred host cells for transformation of vectors and expression of antigen-binding proteins of the present invention are mammalian cells, e.g., COS-7 cells, chinese hamster ovary (CHO) cells, and cell lines of lymphoid origin such as lymphoma, myeloma, or hybridoma cells. Other eukaryotic host, such as yeasts are alternatively used. The transformed host cells are cultured by methods known in the art in a liquid medium containing assimilable sources of carbon, e.g. carbohydrates such as glucose or lactose, nitrogen, e.g. amino acids, peptides, proteins or their degradation products such as peptones, ammonium salts or the like, and inorganic salts, e.g. sulfates, phosphates and/or carbonates of sodium, potassium, magnesium and calcium. The medium furthermore contains, for example, growth-promoting substances, such as trace elements, for example iron, zinc, manganese and the like.

Each variable domain of the antigen-binding proteins of the present invention may be a complete immunoglobulin heavy or light chain variable domain, or it may be a functional equivalent or a mutant or derivative of a naturally occurring domain, or a synthetic domain constructed, for example, in vitro using a technique such as one described in WO 93/11236 (Medical Research Council et al./Griffiths et al.). For instance, it is possible to join together domains corresponding to antibody variable domains which are missing at least one amino acid. The important characterizing feature is the ability of each variable domain to associate with a complementary variable domain to form an antigen binding site.

Similarly, an important feature of constant domains is the ability to form a stable complex. Although antigen binding proteins of the invention comprise complete C_{L} and C_{H}1 domains, the invention also contemplates the use of modified C_{L} and C_{H}1 domains which may have amino acids deleted or inserted, and which may or may not have an interchain disulfide bond, so long as the domains can associate in a stable complex.

Important characterizing features of Fc constant domains include the ability to self-associate, to bind to an Fc receptor, to initiate CMC and to initiate ADCC. As previously noted, antigen-binding protein of the invention do not require that every constant domain structure or function be present. Accordingly, the terms heavy chain variable domain, light chain variable domain, constant domain, scFv and Fc should be construed to include all variants which are functionally equivalent.

In a preferred embodiment of the invention, the antigen binding sites of a bispecific antibody comprise scFv domains having two different binding specificities. For example, substituted for the V_{L} and V_{H} domains of an IgG molecule are scFv domains of different specificity such that the resulting molecule, herein designated Bs(scFv)4-IgG, is bivalent for each of its target antigens. Bs(scFv)4-IgG is functionally expressed and assembled in a variety of expression systems, and particularly in mammalian cells, and is capable of binding to two different epitopes simultaneously.

As provided previously herein, a scFv is preferred for linkage to light chain and heavy chain constant domains. However, where desired or convenient the structure comprising the antigen binding site of a bispecific antigen binding protein of the invention includes more or less than an Fv. For example, it further includes constant region portions (e.g., linkage of an Fab to a light chain or heavy chain domain) or only a portion of an Fv (e.g., where antigen binding is determined predominantly by one variable domain and the second variable domain contributes little to affinity or specificity). Thus, an antigen binding site comprises of a single polypeptide chain which is further linked to a light chain or heavy chain constant region, allowing the arrangement of domains in the antigen-binding protein to be unambiguously predetermined, and to form an overall Ig-form structure with at least two constant domains.

An antigen binding site for inclusion in a antigen-binding protein having desired binding characteristics is obtained by a variety of methods. The amino acid sequences of the V_{L} and V_{H} portions of a selected binding domain correspond to a naturally-occurring antibody or are chosen or modified to obtained desired immunogeinc or binding characteristics. For example, chimeric variable domains are constructed in which antigen binding site derived from a non-human source are substituted into human variable domains. A chimeric construct is particularly valuable for elimination of adverse immunogenic characteristics, for example, where an antigen binding domain from a non-human source is desired to be used for treatment in a human. A preferred chimeric domain is one which has amino acid sequences which comprise one or more complementarity determining regions (CDRs) of a non-human origin grafted to human framework regions (FRs). For examples of such chimeras, see: Jones, P. T. et al., (1996) Nature 321, 522-525; Riechman, L. et al., (1988) Nature 332, 323-327; U.S. Pat. No. 5,530,101 to Queen et al. Variable domains have a high degree of structural homology, allowing easy identification of amino acid residues within variable domains which corresponding to CDRs and FRs. See, e.g., Kabat, E. A., et al. (1991) Sequences of Proteins of Immunological Interest. 5th ed. National Center for Biotechnology Information, National Institutes of Health, Bethesda, Md. Thus, amino acids which participate in antigen binding are easily identified. In addition, methods have been developed to preserve or to enhance affinity for antigen of chimeric binding domains comprising grafted CDRs. One way is to include in the chimeric domain the foreign framework residues which influence the conformation of the CDR regions. A second way is to graft the foreign CDRs onto human variable domains with the closest homology to the foreign variable region. Queen, C. et al., (1989) Proc. Natl. Acad. Sci. USA 86, 10029-10033. CDRs are most easily grafted onto different FRs by first amplifying individual FR sequences using overlapping primers which include desired CDR sequences, and joining the resulting gene segments in subsequent amplification reactions. Grafting of a CDR onto a different variable domain can further involve the substitution of amino acid residues which are adjacent to the CDR in the amino acid sequence or packed against the CDR in the folded variable domain structure which affect the conformation of the CDR. Humanized domains of the invention therefore include human antibodies which comprise one or more non-human CDRs as well as such domains in which additional substitutions or replacements have been made to preserve or enhance binding characteristics.

Chimeric binding domains of the invention also include antibodies which have been humanized by replacing surface-exposed residues to make the scFv appear as self to the immune system (Padlan, E. A. (1991) Mol. Immunol. 28, 489-498). Antibodies have been humanized by this process with no loss of affinity (Roguska et al. (1994) Proc. Natl. Acad. Sci. USA 91, 969-973). Because the internal packing of amino acid residues in the vicinity of the antigen binding site remains unchanged, affinity is preserved. Substitution of surface-exposed residues of a scFv according to the invention for the purpose of humanization does not mean substitution of CDR residues or adjacent residues which influence binding characteristics.

The invention contemplates binding domains which are essentially human. Human binding domains are obtained from phage display libraries wherein combinations of human heavy and light chain variable domains are displayed on the surface of filamentous phage (See, e.g., McCafferty et al. (1990) Nature 348, 552-554; Aujame et al. (1997) Human Antibodies 8, 155-168). Combinations of variable domains are typically displayed on filamentous phage in the form of Fabs or scFvs. The library is screened for phage bearing combinations of variable domains having desired antigen binding characteristics. Preferred variable domain combinations display high affinity for a selected antigen and little cross-reactivity to other related antigens. By screening very large repertoires of antibody fragments, (see e.g., Griffiths et al. (1994) EMBO J. 13, 3245-3260) a good diversity of high affinity Mabs are isolated, with many expected to have sub-nanomolar affinities for the desired antigen.

Alternatively, human binding domains can be obtained from transgenic animals into which unrearranged human Ig gene segments have been introduced and in which the endogenous mouse Ig genes have been inactivated (reviewed in Brüggemann and Taussig (1997) Curr. Opin. Biotechnol. 8, 455-458). Preferred transgenic animals contain very large contiguous Ig gene fragments that are over 1 Mb in size (Mendez et al. (1997) Nature Genet. 15, 146-156) but human Mabs of moderate affinity can be raised from transgenic animals containing smaller gene loci (See, e.g., Wagner et al. (1994) Eur. J. Immunol. 42, 2672-2681; Green et al. (1994) Nature Genet. 7, 13-21).

Binding domains of the invention include those for which binding characteristics have been improved by direct mutation or by methods of affinity maturation. Affinity and specificity may be modified or improved by mutating CDRs and screening for antigen binding sites having the desired characteristics (See, e.g., Yang et al. (1995) J. Mol. Bio. 254, 392-403). CDRs are mutated in a variety of ways. One way is to randomize individual residues or combinations of residues so that in a population of otherwise identical antigen binding sites, all twenty amino acids are found at particular positions. Alternatively, mutations are induced over a range of CDR residues by error prone PCR methods (See, e.g., Hawkins et al. (1992) J. Mol. Bio. 226, 889-896). Phage display vectors containing heavy and light chain variable region genes are propagated in mutator strains of E. coli (See, e.g., Low et al. (1996) J. Mol. Bio. 250, 359-368). These methods of mutagenesis are illustrative of the many methods known to one of skill in the art.

In another aspect of the invention, the antigen-binding proteins can be chemically or biosynthetically linked to anti-tumor agents or detectable signal-producing agents. Anti-tumor agents linked to an antibody include any agents which destroy or damage a tumor to which the antibody has bound or in the environment of the cell to which the antibody has bound. For example, an anti-tumor agent is a toxic agent such as a chemotherapeutic agent or a radioisotope. Suitable chemotherapeutic agents are known to those skilled in the art and include anthracyclines (e.g. daunomycin and doxorubicin), methotrexate, vindesine, neocarzinostatin, cis-platinum, chlorambucil, cytosine arabinoside, 5-fluorouridine, melphalan, ricin and calicheamicin. The chemotherapeutic agents are conjugated to the antibody using conventional methods (See, e.g., Hermentin and Seiler (1988) Behring Inst. Mitt. 82, 197-215).

Detectable signal-producing agents are useful in vivo and in vitro for diagnostic purposes. The signal producing agent produces a measurable signal which is detectible by external means, usually the measurement of electromagnetic radiation. For the most part, the signal producing agent is an enzyme or chromophore, or emits light by fluorescence, phosphorescence or chemiluminescence. Chromophores include dyes which absorb light in the ultraviolet or visible region, and can be substrates or degradation products of enzyme catalyzed reactions.

The invention further contemplates antigen-binding proteins of the invention to which target or reporter moieties are linked. Target moieties are first members of binding pairs. Anti-tumor agents, for example, are conjugated to second members of such pairs and are thereby directed to the site where the antigen-binding protein is bound. A common example of such a binding pair is adivin and biotin. In a preferred embodiment, biotin is conjugated to an antigen-binding protein of the invention, and thereby provides a target for an anti-tumor agent or other moiety which is conjugated to avidin or streptavidin. Alternatively, biotin or another such moiety is linked to an antigen-binding protein of the invention and used as a reporter, for example in a diagnostic system where a detectable signal-producing agent is conjugated to avidin or streptavidin.

Suitable radioisotopes for use as anti-tumor agents are also known to those skilled in the art. For example, ¹¹³I or ²¹¹At is used. These isotopes are attached to the antibody using conventional techniques (See, e.g., Pedley et al. (1993) Br. J. Cancer 68, 69-73). Alternatively, the anti-tumor agent which is attached to the antibody is an enzyme which activates a prodrug. In this way, a prodrug is administered which remains in its inactive form until it reaches the tumor site where it is converted to its cytotoxin form once the antibody complex is administered. In practice, the antibody-enzyme conjugate is administered to the patient and allowed to localize in the region of the tissue to be treated. The prodrug is then administered to the patient so that conversion to the cytotoxic drug occurs in the region of the tissue to be treated. Alternatively, the anti-tumor agent conjugated to the antibody is a cytokine such as interleukin-2 (IL-2), interleukin-4 (IL-4) or tumor necrosis factor alpha (TNF-α). The antibody targets the cytokine to the tumor so that the cytokine mediates damage to or destruction of the tumor without affecting other tissues. The cytokine is fused to the antibody at the DNA level using conventional recombinant DNA techniques.

The proteins of the invention can be fused to additional amino acid residues such as a peptide tag to facilitate isolation or purification, or a signal sequence to promote secretion or membrane transport in any particular host in which the protein is expressed.

Specific examples of the invention are provided herein which relate to bispecific proteins having binding domains specific for two different epitopes of KDR and demonstrate the advantageous functional aspects of antigen-binding proteins of the invention. The employed binding domains are derived from scFv p1C11 and scFv p4G7, which are isolated from a phage display library constructed from a mouse immunized with KDR. (Zhu et al., 1998; Lu et al., 1999).

scFv p4G7 binds to an epitope common to both KDR and the mouse homolog Flk-1 and does not interfere with the binding of VEGF to either receptor. scFvp1C11 binds to a separate epitope of KDR and is capable of blocking binding of VEGF, but does not bind to Flk-1. Thus, a bispecific bivalent immunoglobulin-like molecule displaying two of each binding domain is tetravalent for binding to KDR and bivalent for binding to Flk-1.

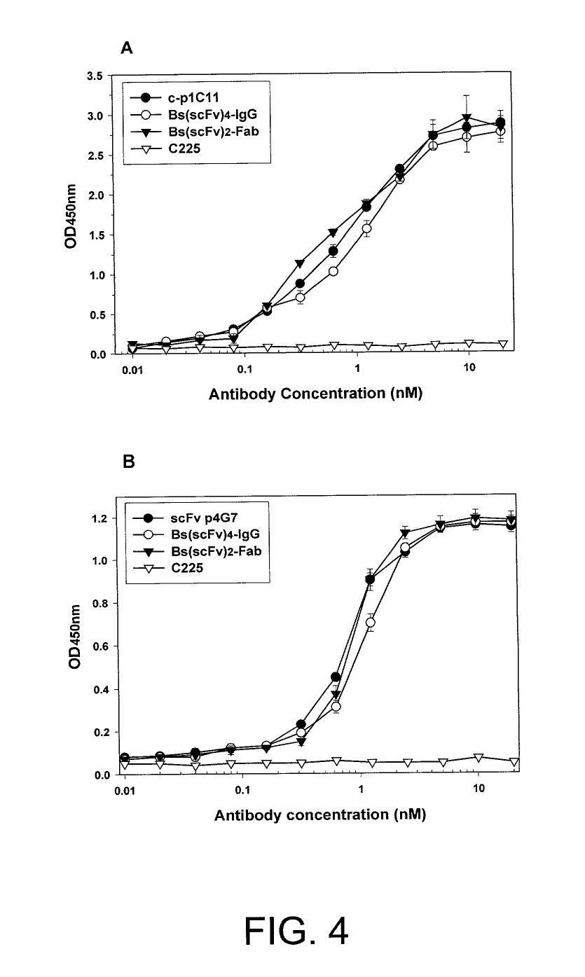

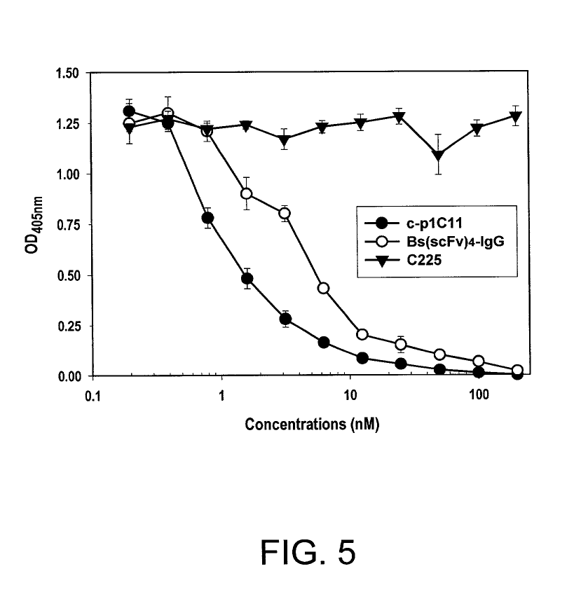

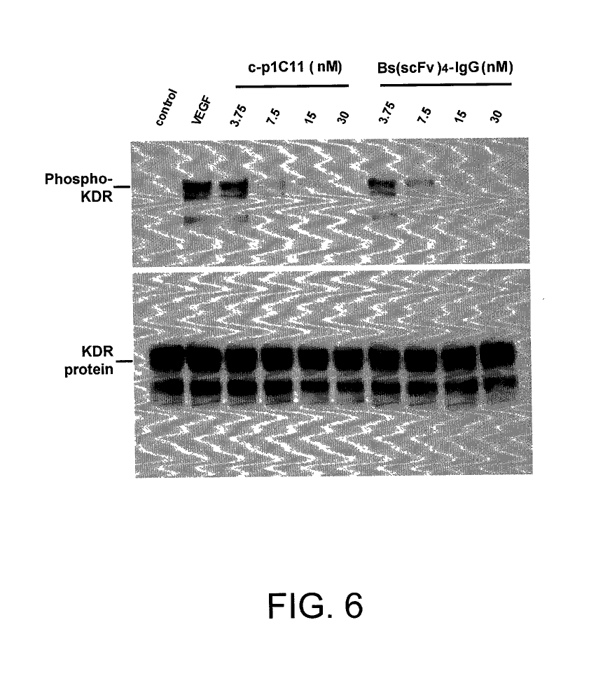

Bs(scFv)4-IgG, which is bivalent to Flk-1, has an avidity similar to DAB p4G7, a bivalent diabody to Flk-1. The avidities of Bs(scFv)4-IgG and DAB p4G7 are approximately 10 to 23-fold higher than their respective monovalent counterparts, Bs(scFv)2-Fab and scFv p4G, demonstrating the enhanced binding which results from bivalency. Bs(scFv)4-IgG retains the biological functions of both of its component binding sites, binding as efficiently as the parent antibodies to both KDR and Flk-1 (FIG. 4). Bs(scFv)4-IgG binds to surface-expressed KDR on human endothelial cells, blocks KDR/VEGF interaction, and efficiently neutralizes VEGF-induced KDR receptor phosphorylation in a dose-dependent manner (FIG. 5 and 6). Notably, Bs(scFv)4-IgG is as potent as c-p1C11 in neutralizing VEGF-induced receptor phosphorylation despite the fact that Bs(scFv)4-IgG binds to KDR with a lower affinity than c-p1C11, and is 4-fold less effective in blocking KDR/VEGF interaction in an ELISA assay. The enhanced biological activity of Bs(scFv)4-IgG is attributable to the enhanced binding which results from being tetravalent with respect to KDR. Bs(scFv)4-IgG has the capacity for intra-molecular cross-linking (i.e., cross-linking two epitopes within the same KDR molecule) and/or inter-molecular cross-linking to form a multimolecular complexes on the cell surface.

The antigen-binding proteins of the present invention are useful for treating diseases in humans and other mammals. The antigen-binding proteins are used for the same purposes and in the same manner as heretofore known for natural and engineered antibodies. The present antigen-binding proteins thus can be used in vivo and in vitro for investigative, diagnostic or treatment methods which are well known in the art.

It is understood that antigen binding proteins of the invention, where used in the human body for the purpose of diagnosis or treatment, will be administered in the form of a composition additionally comprising a pharmaceutically-acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of this invention may be in a variety of forms. These include, for example, solid, semi-solid and liquid dosage forms, such as tablets, pills, powders, liquid solutions, dispersions or suspensions, liposomes, suppositories, injectable and infusible solutions. The preferred form depends on the intended mode of administration and therapeutic application. The preferred compositions are in the form of injectable or infusible solutions.

The preferred pharmaceutical compositions of this invention are similar to those used for passive immunization of humans with other antibodies. The preferred mode of administration is parenteral.

It is to be understood and expected that variations in the principles of invention herein disclosed may be made by one skilled in the art and it is intended that such modifications are to be included within the scope of the present invention.

The examples which follow further illustrate the invention, but should not be construed to limit the scope of the invention in any way. Detailed descriptions of conventional methods, such as those employed in the construction of vectors and plasmids, the insertion of genes encoding polypeptides into such vectors and plasmids, the introduction of plasmids into host cells, and the expression and determination thereof of genes and gene products can be obtained from numerous publication, including Sambrook, J. et al., (1989) Molecular Cloning: A Laboratory Manual, 2^{nd} ed., Cold Spring Harbor Laboratory Press. All references mentioned herein are incorporated in their entirety.

### EXAMPLE 1

### Materials and Methods

Proteins and Antibodies

The complete KDR coding sequence Vascular endothelial growth factor (VEGF), kinase insert domain-containing receptor-alkaline phosphatase fusion protein (KDR-AP) and its mouse homolog, fetal liver kinase 1 (Flk-1)-AP, are expressed in baculovirus and NIH 3T3 cells, respectively, and purified following the procedures described (Zhu et al., 1998).

The human KDR coding sequence is published (GenBank Accession No. AF035121). KDR extracellular domain (ECD) immunoglobulin (Ig) domain deletion mutants are constructed by PCR cloning, expressed in NIH 3T3 cells and purified as described (Lu et al., (2000) J. Biol. Chem. 275, 14321-14330). The KDR ECD Ig domain deletion mutants have the following structures:

KDR(Ig1-7): the full length KDR ECD containing all seven Ig domains of the receptor (from amino acid Met¹ to Val⁷⁴²);

KDR(Ig1-3): the mutant containing the three N-terminal ECD Ig domains (from amino acid Met¹ to Lys³²⁷); and

KDR(Ig3-7): the mutant containing KDR ECD Ig domain 3 through 7 (from amino acid Asp²²⁵ to Val⁷⁴²).

Anti-KDR single chain Fv (scFv) p1C11 and scFv p4G7 are isolated from a phage display library constructed from a mouse immunized with KDR, as reported in Zhu et al. (1998) Cancer Res., 58, 3209-3214 and Lu et al. (1999) J. Immunol. Methods, 230, 159-171.

Diabody DAB p4G7, a form of bivalent scFv fragment (Holliger et al. (1993) Proc. Natl. Acad. Sci. USA 90, 6444-6448; Zhu et al. (1996) Bio/Technology, 14, 192-196) is constructed from scFv p4G7 as previously described in Zhu et al. (1996) and Lu et al. (1999). c-p1C11, a mouse/human chimeric IgGI antibody constructed from scFv p1C11, and C225, a chimeric IgG1 antibody directed against epidermal growth factor (EGF) receptor, are both produced at ImClone Systems Incorporated (New York, N.Y.). Zhu, et al. (1999).

The hybridoma cell line (ATCC No. PTA-334) producing the anti-Flt-1 antibody, Mab6.12 (IgG1, κ), was established at ImClone Systems Incorporated (New York, N.Y.) from a mouse immunized with a recombinant form of the receptor.

Immunization of Mice and Construction of Single Chain Antibody Phage Display Library

Female BALB/C mice are given two intraperitoneal (i.p.) injections of 10 μg KDR-AP in 200 μl of Ribi Adjuvant System followed by one i.p. injection without RIBI adjuvant over a period of two months. The mice are also given a subcutaneous (s.c.) injection of 10 μg KDR-AP in 200 μl of RIBI at the time of the first immunization. The mice are boosted i.p. with 20 μg of KDR-AP three days before euthanasia. Spleens from donor mice are removed and the cells are isolated. RNA is extracted and mRNA is purified from total RNA of splenocytes. Following reverse transcription, cDNAs corresponding to expressed V_{L} and V_{H} genes are separately amplified. The amplified products can be inserted into a vector designed to accept each gene separately or linked to nucleotides encoding a secretion signal sequence and polypeptide linker (e.g., by PCR amplification) and the fused product inserted into a desired vector. See, e.g., Zhu et al., 1998.

Materials and procedures for displaying mouse scFv on filamentous phage are commercially available (Recombinant Phage Antibody System, Amersham Pharmacia Biotech). Briefly, to display the scFv on filamentous phage surface, antibody V_{H} and V_{L} domains are joined together by a 15 amino acid linker (GGGGS)₃. The C terminus of this construct is joined to the N terminus of phage protein III with a 15 amino-acid E tag, ending with an amber codon (TAG). The amber codon positioned between the E tag and protein III allows production of scFv in soluble form when transformed into a nonsupressor host (e.g., HB2151 cells), and phage display via protein III when transformed into a suppressor host (e.g., TG1 cells).

The scFv-gene III construct is ligated into the pCANTAB 5E vector. Transformed TG1 cells are plated onto 2YTAG plates (17 g/l tryptone, 10 g/l yeast extract, 5 g/l NaCl, 20 g/l glucose, 100 μg/ml ampicillin, 15 g/l Bacto-agar) and incubated. The colonies are scraped into 10 ml of 2YT medium (17 g/l tryptone, 10 g/l yeast extract, 5 g/l NaCl), mixed with 5 ml 50% glycerol and stored at -70° C. as the library stock.

Biopanning

The library stock is grown to log phase, rescued with M13K07 helper phage and amplified overnight in 2YTAK medium (2YT containing 100 μg/ml of ampicillin and 50 μg/ml of kanamycin) at 30° C. The phage preparation is precipitated in 4% PEG/0.5M NaCl, resuspended in 3% fat-free milk/PBS containing 500 μg/ml of alkaline phosphatase (AP) and incubated at 37° C. for 1 h to block phage-scFv having specificity for AP scFv and to block other nonspecific binding.

KDR-AP (10 μg/ml) coated Maxisorp Star tubes (Nunc, Denmark) are first blocked with 3% milk/PBS at 37° C. for 1 h, and then incubated with the phage preparation at room temperature for 1 h. The tubes are washed 10 times with PBST (PBS containing 0.1% Tween 20), followed by 10 times with PBS. The bound phage is eluted at room temperature for 10 min. with 1 ml of a freshly prepared solution of 100 mM triethylamine. The eluted phage are incubated with 10 ml of mid-log phase TG1 cells at 37° C. for 30 min. stationary and 30 min. shaking. The infected TG1 cells are then plated onto 2YTAG plates and incubated overnight at 30° C. as provided above for making of the phage stock.

Successive rounds of the screening procedure (panning) are employed to further enrich for displayed scFv having the desired binding specificity. After two or three rounds of panning, individual bacterial colonies are screened individually to identify clones having desired KDR binding characteristics. Identified clones can be further tested for blocking of VEGF binding. DNA fingerprinting of clones is used to differentiate unique clones. Representative clones of each digestion pattern are picked and subject to DNA sequencing.

Phage ELISA

Individual TG1 clones are grown at 37° C. in 96 well plates and rescued with M13K07 helper phage as described above. The amplified phage preparation is blocked by addition of 1/6 volume of 18% milk/PBS at RT for 1 h and added to Maxi-sorp 96-well microtiter plates (Nunc) which have been coated with KDR-AP or AP (1 μg/ml×100 μl). After incubation at room temperature for 1 h, the plates are washed 3 times with PBST and incubated with a rabbit anti-M13 phage Ab-HRP conjugate. The plates are washed 5 times, TMB peroxidase substrate added, and the OD at 450 nm read using a microplate reader.

Preparation of Soluble scFv

Phage of individual clones are used to infect a nonsuppressor E. coli host HB2151 and the infectant selected on 2YTAG-N (2YTAG; 100 μg/ml nalidixic acid) plates. Expression of scFv in HB2 151 cells is induced by culturing the cells in 2YTA medium containing 1 mM isopropyl-1-thio-B-D-galactopyranoside at 30° C. A periplasmic extract of the cells is prepared by resuspending the cell pellet in 25 mM Tris (pH 7.5) containing 20% (w/v) sucrose, 200 mM NaCl, 1 mM EDTA and 0.1 mM PMSF, followed by incubation at 4° C. with gentle shaking for 1 h. After centrifugation at 15,000 rpm for 15 min., the soluble scFv is purified from the supernatant by affinity chromatography using the RPAS Purification Module (Pharmacia Biotech).

Preparation of scFv from Mab6.12

The V_{H} and V_{L} genes of Mab 6.12 are cloned by RT-PCR from mRNA isolated from the hybridoma cells, following the procedures of Bendig et al. (1996) In: Antibody Engineering: A Practical Approach, McCafferty, J., Hoogenboom, H.R., Chiswell, D. J., eds., Oxford University Press, Incorporated; p147-168. Eleven 5′ primers, specifically designed to hybridize to the 5′ ends of mouse antibody light chain leader sequences, and one 3′ primer that hybridizes to the 5′ end of mouse κ light chain constant region, are used to clone the V_{L} gene. Twelve 5′ primers, specifically designed to hybridize to the 5′ ends of mouse antibody heavy chain leader sequences, and one 3′ primer that hybridizes to the 5′ end of mouse IgG1 heavy chain constant region are used to clone the V_{H} gene. In total, twenty-three PCR reactions, eleven for the V_{L} gene and twelve for the V_{H} gene, are carried out for each of the antibodies. All PCR-generated fragments with size between 400 to 500 base pairs are cloned into the pCR® 2.1 vector as described in the manufacturer's instruction (TA Cloning® Kit, Invitrogen, Carlsbad, Calif.), followed by transformation of E. coli strain, XL-1.

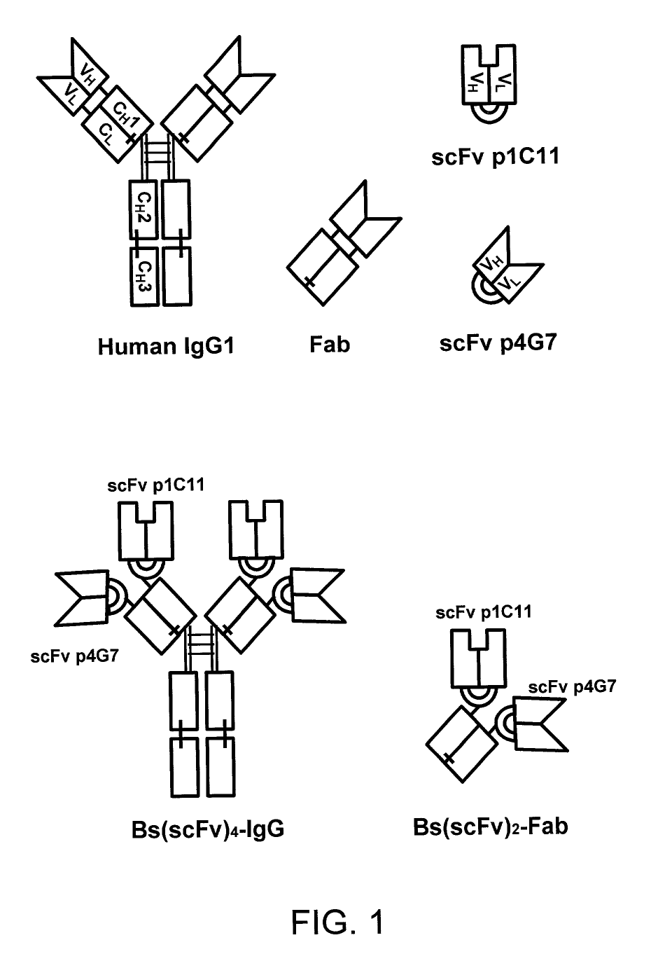

PCR fragments encoding the V_{L} and the V_{H} genes of MAB 6.12 are used to assemble scFv 6.12, using overlapping PCR. In this scFv, the C-terminal of Mab 6.12 V_{H} is linked to the N-terminal of Mab 6.12 V_{L} via a 15 amino acid linker, (Glycine-Glycine-Glycine-Glycine-Serine)₃, or (GGGGS)₃ (FIG. 1A). The scFv 6.12-encoding gene is then cloned into vector pCANTAB 5E (Amersham Pharmacia Biotech, Piscataway, N.J.) for the expression of the soluble scFv protein.

Construction of Expression Vectors for BsAb-IgG [Bs(scFv)4-IgG] and BsAb-Fab[Bs(svFv)2-Fab]

A gene encoding scFv p4G7 is amplified from the scFv expression vector by PCR using primers JZZ-2 (SEQ ID NO: 29) and JZZ-3 (SEQ ID NO: 30). A leader peptide sequence for protein secretion in mammalian cells is then added to the 5′ end of the scFv coding sequence by PCR using primers JZZ-12 (SEQ ID NO: 31) and JZZ-3 (SEQ ID NO: 30).

Similarly, the gene encoding scFv p1C11 is amplified from the scFv expression vector by PCR using primers JZZ-2 (SEQ ID NO: 29) and p1C11V L3-2 (SEQ ID NO: 32), followed by PCR with primers JZZ-12 (SEQ ID NO: 31) and p1C11VL3-2 (SEQ ID NO: 32) to add the leader peptide sequence.

The same leader peptide consisting of 19 amino acids, MGWSCIILFLVATATGV_{H}S (SEQ ID NO: 33), is used for secretion of both the light and the heavy chains.

Separate expression vectors for the light and heavy chains of Bs(scFv)4-IgG are constructed. The cloned scFv p4G7 gene is digested with Hind III and BamHI and ligated into the vector pKN100 (a gift from Dr. S. T. Jones, MRC Collaborative Center, London, England) containing the human K light chain constant region (C_{L}) to create the expression vector for the BsAb-IgG light chain, BsIgG-L. The cloned scFv p1C11 gene is digested with Hind III and BamHI and ligated into the vector pGD1D105 (a gift from Dr. S. T. Jones) containing the human IgG1 heavy chain constant domain (C_{H}) to create the expression vector for the BsAb-IgG heavy chain, BsIgG-H. These vectors are similar to the light chain (HCMV-V_{L}-HC_{K}) and heavy chain (HCMV-V_{H}-HC_{γ1}) vectors described in U.S. Pat. 5,840,299 except for the presence of a DHFR gene which confers resistance to methotrexate and provides amplification of vector sequences.

To prepare the expression vector for Bs(scFv)2-Fab, a stop codon is introduced into vector BsIgG-H immediately after the first constant domain (C_{H}1) to terminate the protein translation, by PCR using primers JZZ-12 (SEQ ID NO: 31) and JZZ-18 (SEQ ID NO: 34). The gene fragment is digested with Hind III and NaeI and inserted into vector pG1D105 to create vector BsFab-H. All constructs are examined by restriction enzyme digestion and verified by DNA sequencing.

The primer sequences used in this example are provided below and in the Sequence

!Listing.
JZZ-2 Sequence (SEQ ID NO: 29):
5′-CTAGTAGCAACTGCCACCGGCGTACATTCACAGGTCAAGCTGC-3′
JZZ-3 Sequence (SEQ ID NO: 30):
5′-TCGAAGGATCACTCACCTTTTATTTCCAGC-3′
JZZ-12 Sequence (SEQ ID NO: 31):
5′-GGTCAAAAGCTTATGGGATGGTCATGTATCATCCTTTTTCTAGTAGCAACT-3′
p1C11VL3-2 Sequence (SEQ ID NO: 32):
5′-TCGATCTAGAAGGATCCACTCACGTTTTATTTCCAG-3′
Leader Peptide (SEQ ID NO: 33):
MGWSCIILFLVATATGVHS
JZZ-18 (SEQ ID NO: 34):
5′-TCTCGGCCGGCTTAAGCTGCGCATGTGTGAGT-3′

Antibody Expression and Purification

COS cells are co-transfected with equal amounts of DNA from vector BsIgG-L and BsIgG-H, or BsIgG-L and BsFab-H, for transient expression of Bs(scFv)4-IgG and Bs(scFv)2-Fab, respectively, following the procedure described in Zhu et al. (1999) Cancer Lett. 136, 203-213. The cells are switched to serum-free medium 24 h after transfection. The conditioned supernatant is collected at 48 h and 120 h after transfection. The Bs(scFv)4-IgG and Bs(scFv)2-Fab are purified from the pooled supernatant by affinity chromatography using Protein G column following the protocol described by the manufacturer (Pharmacia Biotech, Piscataway, N.J.). The antibody-containing fractions are pooled, buffer exchanged into PBS and concentrated using Centricon 10 concentrators (Amicon Corp., Beverly, Mass.). The purity of the antibodies is analyzed by SDS-PAGE. The concentration of purified antibody is determined by ELISA using goat anti-human IgG Fc specific antibody as the capture agent and HRP-conjugated goat anti-human K chain antibody as the detection agent. A standard curve is calibrated using clinical grade antibodies, C225 or c-p1C11.

Binding Assays for Bispecific Antibodies to KDR

Two different assays are carried out to demonstrate the dual specificity of the BsAb described hereinabove.

In the direct binding assay, a 96-well plate (Nunc, Roskilde, Denmark) is first coated with KDR(Ig1-7)-AP, KDR(Ig1-3)-AP or KDR(Ig3-7)-AP fusion proteins (1.0 μg/ml×100 μl per well) using a rabbit anti-AP antibody (DAKO-Immunogloblins A/S, Denmark) as the capturing agent. The plate is then incubated with the BsAb, c-p1C11 or DAB p4G7 at room temperature for 1 h, followed by incubation with rabbit anti-human IgG Fc specific antibody-HRP conjugate (Cappel, Organon Teknika Corp. West Chester, Pa.) for the BsAb and c-p1C11 or mouse anti-E tag antibody-HRP conjugate (Pharnacia Biotech) for DAB p4G7. The plates are washed five times, TMB peroxidase substrate (KPL, Gaithersburg, Md.) is added and the OD at 450 nm read using a microplate reader (Molecular Device, Sunnyvale, Calif.) (Zhu et al., 1998).

In the cross-linking assay, the antibodies are first incubated in solution with KDR(Ig1-7)-AP, KDR(Ig1-3)-AP or KDR(Ig3-7)-AP. The mixtures are transferred to a 96-well plate coated with KDR(Ig1-3) (untagged) and incubated at room temperature for 2 h. The plate is washed and the KDR(Ig1-3) (untagged)-bound AP activity is measured by the addition of AP substrate, p-nitrophenyl phosphate (Sigma) and read OD at 405 nm (Zhu et al., 1998).

Quantitative Binding Assay for Bs(scFv)4-IgG and Bs(scFv)2-Fab to KDR and Flk-1

Various amounts of Bs(scFv)4-IgG, Bs(scFv)2-Fab, c-plCl 1 or scFv p4G7 are added to 96-well Maxi-sorp microtiter plates (Nunc) coated with either KDR-AP or Flk-1-AP (100 ng protein/well) and incubated at room temperature for 1 h, followed by incubation at room temperature for 1 h with rabbit anti-human IgG Fc specific antibody-HRP conjugate for bispecific antibodies and c-p1C11 or mouse anti-E tag antibody-HRP conjugate for scFv p4G7. The plates are washed and developed as described above.

Flow Cytometry (FA CS) Analysis

Early passage HUVEC cells are grown in growth factor-depleted EBM-2 medium overnight to induce the expression of KDR receptor. The cells are harvested and washed three times with PBS, incubated with 5 μg/ml Bs(scFv)4-IgG or c-plCl 1 for 1 h at 4° C., followed by incubation with a FITC-labeled rabbit anti-human Fc antibody (Cappel, Organon Teknika Corp.) for an additional 1 h. The cells are washed and analyzed by a flow cytometer (Zhu et al., 1999).

Analysis of Binding Kinetics

The binding kinetics of the BsAb and parent scFv are measured by surface plasmon resonance, using a BlAcore biosensor (Pharmacia Biosensor). KDR-AP, Flk-1-AP, or Flt-1-Fc fusion proteins are immobilized onto a sensor chip, and various antibodies are injected at concentrations ranging from 1.5 nM to 200 nM. Sensorgrams are obtained at each concentration and are evaluated using a program, BIA Evaluation 2.0, to determine the rate constants kₒₙ and k_{off.} Kd is calculated as the ratio of rate constants k_{off}/kₒₙ.

VEGF/KDR, VEGF/Flt-1. and PIGF/Flt-1 Ligand Blocking Assays

In the blocking assay, various amounts of BsAb, scFv or c-p1C11 are mixed with a fixed amount of KDR-AP, Flk-1-AP or Flt-1-Fc (R&D Systems, Minneapolis, Minn.) and incubated at room temperature for 1 h. The mixtures are then transferred to VEGF₁₆₅₋ or PlGF-coated 96-well plates and incubated at RT for an additional 2 h after which the plates are washed 5 times. VEGF₁₆₅ and PlGF are typically coated at 200 ng/well. VEGF165 is the 165 amino acid form of VEGF. For KDR-AP or Flk-1-AP, the VEGF-bound AP activity is quantified as described (Zhu, et al., 1998; 1999). To determine VEGF- or PlGF-bound Flt-1-Fc, the plate is incubated with a mouse anti-human Fc-HRP conjugate.

Phosphorylation Inhibition Assay

The KDR phosphorylation assay is carried out following the procedure previously described (Zhu et al., 1998; 1999), using a stable 293 cell line transfected with the full length KDR (ImClone Systems). Briefly, the transfected 293 cells (˜3×10⁶ cells per plate) are incubated in the presence or absence of antibodies for 15 min, followed by stimulation with 20 ng/ml of VEGF165 at room temperature for an additional 15 min. The cells are then lysed and the cell lysate used for KDR phosphorylation assays. The KDR receptor is immunoprecipitated from the cell lysates with Protein A Sepharose beads (Santa Cruz Biotechnology, Inc., Calif.) coupled to an anti-KDR antibody, Mab 4.13 (ImClone Systems). Proteins are resolved with SDS-PAGE and subjected to Western blot analysis. To detect KDR phosphorylation, blots are probed with an anti-phosphotyrosine Mab, PY20 (ICN Biomedicals, Inc. Aurora, Ohio). The signals are detected using enhanced chemi-luminescence (Amersham, Arlington Heights, Ill.). The blots are reprobed with a polyclonal anti-KDR antibody (ImClone Systems) to assure that an equal amount of protein is loaded in each lane of the SDS-polyacrylamide gels.

Anti-mitogenic Assay

HUVEC (5×10³ cells/well) are plated onto 96-well tissue culture plates (Wallach, Inc., Gaithersburg, Md.) in 200 ul of EBM-2 medium (Clonetics, Walkersville, Md.) without VEGF, basic fibroblast growth factor (bFGF) or epidermal growth factor (EGF) and incubated at 37° C. for 72 h. Various amounts of antibodies are added to duplicate wells and pre-incubated at 37° C. for 1 h, after which VEGF165 is added to a final concentration of 16 ng/ml. After 18 h of incubation, 0.25 uCi of [³H]-thymidine ([³H]-TdR) (Amersham) is added to each well and incubated for an additional 4 h. The cells are placed on ice, washed twice with serum-containing medium, followed by a 10 minute incubation at 4° C. with 10% TCA. The cells are then washed once with water and solubilized in 25 μl of 2% SDS. Scintillation fluid (150 μl/well) is added and DNA incorporated radioactivity is determined with a scintillation counter (Wallach, Model 1450 Microbeta Scintillation Counter).

Leukemia Migration Assay

HL60 and HEL cells are washed three times with serum-free plain RPMI 1640 medium and suspended in the medium at 1×10⁶/ml. Aliquots of 100 μl cell suspension are added to either 3-μm-pore transwell inserts (for HL60 cells), or 8-,um-pore transwell inserts (for HEL cells) (Costar®, Corning Incorporated, Corning, N.Y.) and incubated with the antigen binding proteins for 30 min at 37° C. The inserts are then placed into the wells of 24-well plates containing 0.5 ml of serum-free RPMI 1640 with or without VEGF₁₆₅. The migration is carried out at 37° C., 5% CO₂ for 16-18 h for HL60 cells, or for 4 h for HEL cells. Migrated cells are collected from the lower compartments and counted with a Coulter counter (Model ZI, Coulter Electronics Ltd., Luton, England).

### EXAMPLE 2

### Production of Bispecfic Antibodies

Construction of Bs(scFv)4-IgG and Bs(scFv)2-Fab

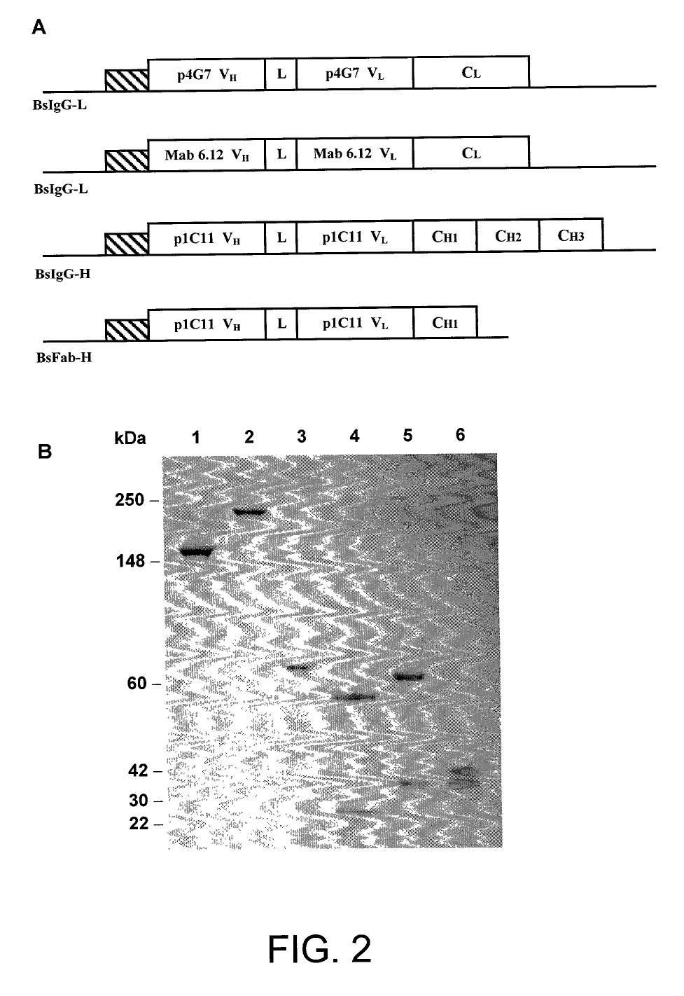

Two anti-KDR scFv antibodies, scFv plC 11 and p4G7, are used for the construction of Bs(scFv)4-IgG and Bs(scFv)2-Fab (FIG. 2A). ScFv p1C11 binds specifically to KDR and blocks KDRIVEGF interaction, whereas scFv p4G7 binds to both KDR and its mouse homolog, Flk-1, but does not block either KDR/VEGF or Flk-1/VEGF interaction (Zhu et al., 1998, Lu et al., 1999). Epitope mapping studies reveal that p1C11 binds to epitope(s) located within KDR ECD Ig domain 1 to 3, whereas the epitope(s) for p4G7 are located within Ig domain 6 and 7 (Lu et al., 2000). Gene segments encoding scFv p1C11 and p4G7 are joined to gene segments encoding C_{H and C}_{L} of a human IgG1 molecule, respectively, so that the scFv sequences are fused to the N-terminal end of C_{H}1 and C_{L}, respectively, to create expression vectors BsJgG-H and BsIgG-L (FIG. 2A). This arrangement replaces the original V_{H} and V_{L} domains of an IgG with two scFv molecules, each constituting an independent antigen-binding unit (FIG. 1). Co-expression of BsIgG-H and BsIgG-L yields an IgG-like bivalent, bispecific molecule, Bs(scFv)4-IgG (FIG. 1). A monovalent, bispecific Fab-like molecule (FIG. 1), Bs(scFv)2-Fab, is also produced by co-expression of BsIgG-L and BsFab-H. Vector BsFab-H is constructed from BsIgG-H by introducing a stop codon at the end of C_{H}1 domain (FIG. 2A).

Expression and Purification of Bs(scFv)4-IgG and Bs(scFv)2-Fab

The Bs(scFv)4-IgG and Bs(scFv)2-Fab are transiently expressed in COS cells and purified from the cell culture supernatant by an affinity chromatography using a Protein G column. The purified BsAb is analyzed by SDS-PAGE (FIG. 2B). Under non-reducing condition, Bs(scFv)4-IgG gives rise to a single band with a molecular mass of approximately 200 kDa, whereas Bs(scFv)2-Fab gives a major band of ˜75 kDa (FIG. 2B, lanes 2 and 3). Under reducing conditions, Bs(scFv)4-IgG yields two major bands with the expected mobility for scFv-CH1-CH2-CH3 fusion (˜63 kDa) and scFv-C_{L} fusion (˜37 kDa), respectively (FIG. 2B, lane 5). On the other hand, Bs(scFv)2-Fab gives rise to two major bands with molecular mass of ˜38 kDa and 37 kDa, representing the scFv-C_{H}1 and scFv-C_{L} fusions, respectively (FIG. 2B, lane 6). As a control, c-p1C11, a chimeric IgGI antibody, gives rise to one band of ˜150 kDa under non-reducing conditions (FIG. 2B, lane 1) and two bands of ˜50 kDa (the heavy chain, V_{H}-C_{H}1 -C_{H}2-C_{H}3 fusion) and ˜25 kDa (the light chain, V_{L}-C_{L} fusion) under reducing conditions (FIG. 2B, lane 5).

### EXAMPLE 3

### BsAb Simultaneously Bind to Two Epitopes

Dual Specificity of the BsAb

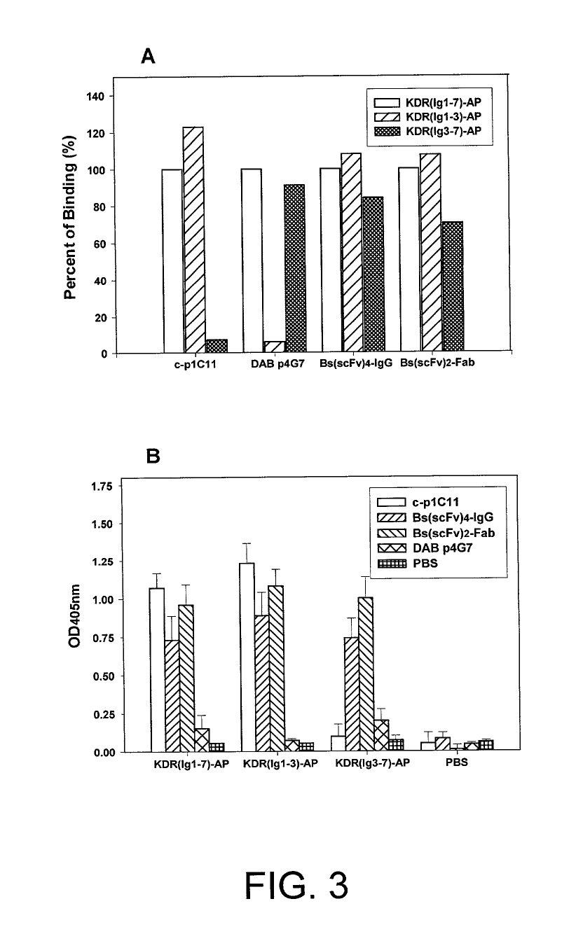

Dual specificity of the BsAb is assayed using the full length KDR ECD and two of its Ig domain-deletion mutants (FIG. 3A). As previously seen, p1C11 only binds to KDR mutants containing Ig domain 1 to 3 (Zhu et al., 1999), whereas p4G7 only binds to mutants containing Ig domain 6 and 7 (Lu et al., 1999). In contrast, both Bs(scFv)4-IgG and Bs(scFv)2-Fab bind to all three KDR variants, indicating that the BsAbs possess two binding sites; one to the epitope on Ig domain 1 to 3 and the other to the epitope on Ig domain 6 and 7.

To investigate whether the BsAb are capable of simultaneous binding to both epitopes, a cross-linking assay is carried out using several KDR ECD Ig domain-deletion mutants that are either untagged or tagged with AP. In this assay, the BsAb are first incubated with KDR(Ig1-7)-AP, KDR(Ig1-3)-AP or KDR(Ig3-7)-AP. The mixtures are transferred to a microtiter plate coated with KDR(Ig1-3) (untagged), followed by measuring KDR(Ig1-3) (untagged)-bound AP activity (FIG. 3B). Both Bs(scFv)4-IgG and Bs(scFv)2-Fab bind effectively to all three KDR-AP variants in solution and form cross-linking complexes with the immobilized KDR(Ig1-3) (untagged), as demonstrated by plate-bound AP activity (FIG. 3B). In contrast, c-p1C11 only cross-links KDR(Ig1-3) (untagged) with KDR variants containing Ig domain 1 to 3, i.e., KDR(Ig1-7)-AP and KDR(Ig1-3)-AP, but not KDR(Ig3-7)-AP. As expected, p4G7 fails to cross-link any KDR variants to the immobilized KDR(Ig1-3) (untagged), since p4G7 does not bind to the KDR(Ig1-3) mutant.

Antigen Binding by BsAb

The antigen binding efficiency of the BsAb is determined on immobilized KDR (FIG. 4A) and Flk-1 (FIG. 4B). FIG. 4A shows the dose-dependent binding of Bs(scFv)4-IgG and Bs(scFv)2-Fab to KDR. Both Bs(scFv)4-IgG and Bs(scFv)2-Fab bind KDR as efficiently as c-p1C11, a chimeric anti-KDR antibody with an affinity 8 to 10 fold greater that p1C11 from which it is derived. Bs(scFv)4-IgG and Bs(scFv)2-Fab, but not c-p1C11, also bind to Flk-1 in a dose-dependent manner similar to scFv p4G7 (FIG. 4B). As expected, C225, a chimeric antibody directed against human EGFR, does not bind to either of the antigens.

Binding of the BsAb to cell surface-expressed receptor is assayed by FACS analysis. As previously seen with c-p1C11 (Zhu et al., 1999), Bs(scFv)4-IgG binds efficiently to KDR expressed on early passage HUVEC.

The binding kinetics of the BsAb to KDR and Flk-1 are determined by surface plasmon resonance using a BlAcore instrument (Table 1). The overall affinities (Kd), or avidities, of Bs(scFv)4-IgG and Bs(scFv)2-Fab to KDR are 1.4 nM and 1.1 nM, respectively, which are similar to those of the monovalent scFv p1C11 and p4G7, but are 4- to 10-fold weaker than those of the bivalent c-p1C11 or DAB p4G7. On the other hand, Bs(scFv)4-IgG, which is bivalent to Flk-1, shows an avidity (Kd, 0.33 nM) that is similar to that of the bivalent DAB p4G7 (Kd, 0.18 nM). Bs(scFv)2-Fab and scFv p4G7, both monovalent to Flk-1, bind to Flk-1 with similar affinity (Kd, 1.7 nM and 4.2 nM, respectively), which are 5 to 20-fold weaker than those of their bivalent counterparts.

VEGF Blocking by Bs(scFv)4-IgG

FIG. 5 shows that Bs(scFv)4-IgG effectively block KDR-AP from binding to immobilized VEGF. The IC50, the antibody concentrations required to block 50% of KDR binding, of Bs(scFv)4-IgG and c-p1C11 are 4 nM, and 1 nM, respectively. As seen with scFv p4G7, Bs(scFv)4-IgG does not block binding of the KDR mouse homolog Flk-1 to VEGF (not shown). Bs(scFv)4-IgG binds to the Flk-1 epitope corresponding to scFv p4G7 which does not affect VEGF/Flk-1 binding. The KDR epitope for which scFv p1c 1 is specific is absent from Flk-1. Thus, VEGF binding to Flk-1 is not blocked. C225, an anti-EGFR antibody, showed no effect on KDR binding to VEGF.

KDR Phosphorylation Inhibition by the BsAb

The biological effect of Bs(scFv)4-IgG on VEGF-induced receptor phosphorylation is determined using KDR-transfected 293 cells. As shown in FIG. 6, VEGF treatment induces strong phosphorylation of KDR receptor. Pre-treatment with Bs(scFv)4-IgG inhibits VEGF-induced receptor phosphorylation in a dose-dependent manner (FIG. 6). Further, Bs(scFv)4-IgG is equally potent as c-p1C11 at each antibody concentration assayed.

Inhibition of Mitogenesis

The effect of anti-KDR antibodies on VEGF-stimulated mitogenesis of human endothelial cells is determined with a [³H]-TdR DNA incorporation assay using HUVEC. HUVEC (5×10^{'}cells/well) are plated into 96-well tissue culture plates in 200 μl of EBM-2 medium without VEGF, bFGF or EGF and incubated at 37° C. for 72 h. Various amounts of antibodies are added to duplicate wells and pre-incubated at 37° C. for 1 hour, after which VEGF165 is added to a final concentration of 16 ng/ml. After 18 hours of incubation, 0.25 μCi of [³H]-TdR is added to each well and incubated for an additional 4 hours. DNA incorporated radioactivity is determined with a scintillation counter.

Both scFv p1C11 and Bs(scFv)4-IgG effectively inhibit mitogenesis of HUVEC stimulated by VEGF. Bs(scFv)4-IgG is a stronger inhibitor of VEGF-induced mitogenesis of HUVEC than the parent scFv. As expected, scFv p2A6, which does not bind KDR, and scFv p4G7, which does not block KDR/VEGF binding, do not show any inhibitory effect on VEGF-stimulated endothelial cell proliferation.

We claim:

1. An antigen-binding protein comprising a complex of two first polypeptides and two second polypeptides, said first polypeptide having an antigen-binding site located to the N terminus of an immunoglobulin light chain constant domain (C_{L} domain), said C_{L} domain capable of stable association with an immunoglobulin heavy chain first constant domain (C_{H}1 domain), and said second polypeptide having an antigen-binding site located to the N terminus of said C_{H}1 domain, said C_{H}1 domain followed by one or more heavy chain constant domains capable of stable self-association.

2. The antigen-binding protein of claim 1 wherein one or more of said antigen-binding sites are provided by a single chain Fv.

3. The antigen-binding protein of claim 1 wherein said antigen-binding sites of said first and second polypeptides have different specificities.

4. The antigen-binding protein of claim 1 wherein said antigen-binding sites of said first and second polypeptides have the same specificity.

5. The antigen-binding protein of claim 3 wherein said specificities are for epitopes which reside on different antigens.

6. The antigen-binding protein of claim 3 wherein said specificities are for epitopes which reside on the same antigen.

7. The antigen-binding protein of claim 1 wherein said first polypeptide and said second polypeptide are covalently bound together.

8. The antigen-binding protein of claim 1 wherein said two second polypeptides are covalently bound together.

9. The antigen-binding protein of claim 1 wherein said second polypeptide has C_{H}1, C_{H}2 and C_{H}3 domains of an antibody of isotype IgA, IgD or IgG.

10. The antigen-binding protein of claim 1 wherein said second polypeptide has C_{H}₁, C_{H}2, C_{H}3 and C_{H}4 domains of an antibody of isotype IgE or IgM.

11. The antigen-binding protein of claim 1 wherein said constant domains are mammalian constant domains.

12. The antigen-binding protein of claim 1 wherein said constant domains are human constant domains.

13. The antigen-binding protein of claim 1 wherein one or more of said single chain Fvs are mouse single chain Fvs.

14. The antigen-binding protein of claim 1 wherein one or more of said single chain Fvs are chimeric single chain Fvs having human framework regions.

15. The antigen-binding protein of claim 1 wherein said single chain Fv has human V_{L} and V_{H} domains.

16. The antigen-binding protein of claim 1 wherein the heavy chain constant domains capable of stable self association are selected from the group consisting of C_{H}2, C_{H}3, and C_{H}4 domains from any immunoglobulin isotype or subtype.

17. The antigen-binding protein of claim 1 which is capable of binding to an Fc receptor.

18. The antigen-binding protein of claim 1 which is capable of effecting complement mediated cytotoxicity (CMC).

19. The antigen-binding protein of claim 1 which is capable of effecting antibody dependent cell-mediated cytotoxicity (ADCC).

20. The antigen-binding protein of claim 1 which is linked to an anti-tumor agent.

21. The antigen-binding protein of claim 1 which is linked to a detectable signal producing agent.

22. The antigen-binding protein of claim 1 which neutralizes activation of a VEGF receptor.

23. The antigen-binding protein of claim 22 wherein the VEGF receptor is mammalian.

24. The antigen-binding protein of claim 22 wherein the VEGF receptor is human.

25. The antigen-binding protein of claim 24 wherein the VEGF receptor is encoded by the flt-1 or flk-1 gene.

26. The antigen-binding protein of claim 1 wherein at least one of the antigen-binding sites is specific for KDR.

27. The antigen-binding protein of claim 1 wherein at least one of the antigen-binding sites is specific for FLT1.

28. The antigen-binding protein of claim 1 wherein at least one of the antigen-binding sites is specific for FLT4.

29. The antigen-binding protein of claim 1 wherein at least one of the antigen-binding sites is specific for EGF-R.

30. The antigen-binding protein of claim 1 wherein at least one of the antigen-binding sites is specific for HER2.

31. The antigen-binding protein of claim 1 wherein at least one of the antigen-binding sites is specific for FGF-R.

32. The antigen-binding protein of claim 1 wherein at least one of the antigen-binding sites is specific for PDGF-R.

33. The antigen-binding protein of claim 1 wherein at least one of the antigen-binding sites is specific for a receptor tyrosine kinase.

34. The antigen-binding protein of claim 1 wherein at least one of the antigen-binding sites is specific for Tek.

35. The antigen-binding protein of claim 1 wherein at least one of the antigen-binding sites is specific for Tie-2.

36. The antigen-binding protein of claim 1 wherein one of the antigen-binding sites is specific for KDR and the other antigen-binding site is specific for FLT1.

37. The antigen-binding protein of claim 1 wherein one of the antigen-binding sites is specific for KDR and the other antigen-binding site is specific for an antigen selected from the group consisting of FLT4, EGF-R, HER2, FGF-R, PDGF-R, Tek and Tie2.

38. The antigen-binding protein of claim 1 wherein one of the antigen-binding sites is specific for EGF-R and the other antigen-binding site is specific for HER2.

39. The antigen-binding protein of claim 1 wherein at least one of the antigen-binding sites is specific for a cell-surface antigen of an immune system effector cell.

40. The antigen-binding protein of claim 39 wherein the immune system effector cell is a T cell, a macrophage, a neutrophil, or an NK cell.

41. The antigen-binding protein of claim 39 wherein the cell-surface antigen is CD3, CD16, CD28, CD32, CD64, an Fc receptor, a cytokine receptor or a lymphokine receptor.

42. The antigen-binding protein of claim 39 wherein the cell-surface antigen is a receptor for a cytokine or lymphokine and wherein an antigen-binding site comprises the amino acid sequence of the cytokine or lymphokine or a portion thereof.

43. The antigen-binding protein of claim 42 wherein the receptor is for IL-2, IL-4, IL-5, GM-CSF or G-CSF.

44. The antigen-binding protein of any one of claims 26, 27, 28, 29, 30, 31, 32, 33, 34 and 35 wherein one of the one of the antigen-binding sites is specific for a cell-surface antigen of an immune system effector cell.

45. The antigen-binding protein of claim 44 wherein the immune system effector cell is a T cell, a macrophage, a neutrophil, of an NK cell.

46. The antigen-binding protein of claim 44 wherein the cell-surface antigen is CD3, CD16, CD28, CD32, CD64, an Fc receptor, a cytokine receptor or a lymphokine receptor.

47. An antigen-binding protein comprising a complex of two first polypeptides and two second polypeptides,
said first polypeptide having a single chain Fv located to the N terminus of an immunoglobulin light chain constant domain (C_{L} domain), said C_{L} domain capable of stable association with an immunoglobulin heavy chain first constant domain (C_{H}1 domain), and
said second polypeptide having a single chain Fv located to the N terminus of said C_{H}1 domain, said C_{H}1 domain followed by one or more heavy chain constant domains capable of stable self-association.

48. The antigen-binding protein of claim 47 wherein said antigen-binding sites of said first and second polypeptides have different specificities.

49. The antigen-binding protein of claim 47 wherein said antigen-binding sites of said first and second polypeptides have the same specificity.

50. The antigen-binding protein of claim 47 which neutralizes activation of KDR.

51. The antigen-binding protein of claim 50 wherein one or both of said single chain Fvs is single chain Fv p1dc11.

52. The antigen-binding protein of claim 50 wherein one or both of said single chain Fvs is single chain Fv p4G7.

53. The antigen-binding protein of claim 47 which neutralizes activation of FLTI.

54. The antigen-binding protein of claim 53 wherein one or both of said single chain Fvs is single chain Fv 6.12.

55. The antigen-binding protein of claim 50 wherein the amino acid sequence of the complementarity determining regions (CDRs) of one or both of said single chain Fv is represented by:
SEQ ID NO: 1 at CDRH1;
SEQ ID NO: 2 at CDRH2;
SEQ ID NO: 3 at CDRH3;
SEQ ID NO: 4at CDRL I;
SEQ ID NO: 5 at CDRL2; and
SEQ ID NO: 6 at CDRL3.

56. The antigen-binding protein of claim 50 wherein the nucleotide sequence encoding the complementarity determining regions (CDRs) of one or both of said single chain Fv is represented by:
SEQ ID NO: 9 for CDRH1;
SEQ ID NO: 10 for CDRH2;
SEQ ID NO: 11 for CDRH3;
SEQ ID NO: 12 for CDRL1;
SEQ ID NO: 13 for CDRL2; and
SEQ ID NO: 14 for CDRL3.

57. The antigen-binding protein of claim 50 wherein the amino acid sequence of the variable domains of one or both of said single chain Fv is represented by:
SEQ ID NO: 7 for the heavy-chain variable domain (V_{H}) and
SEQ ID NO: 8 for the light-chain variable domain (V_{L}).

58. The antigen-binding protein of claim 50 wherein the nucleotide sequence encoding the variable domains of one or both of said single chain Fv is represented by:
SEQ ID NO: 15 for the heavy-chain variable domain (V_{H}); and
SEQ ID NO: 16 for the light-chain variable domain (V_{L}).

59. The antigen-binding protein of claim 50 wherein the amino acid sequence of the complementarity determining regions (CDRs) of one or both of said single chain Fv is represented by:
SEQ ID NO: 1 at CDRH1;
SEQ ID NO: 21 at CDRH2;
SEQ ID NO: 3 at CDRH3;
SEQ ID NO: 4at CDRL1;
SEQ ID NO: 5 at CDRL2; and
SEQ ID NO: 6 at CDRL3.

60. The antigen-binding protein of claim 50 wherein the nucleotide sequence encoding the complementarity determining regions (CDRs) of one or both of said single chain Fv is represented by:
SEQ ID NO: 9 for CDRH1;
SEQ ID NO: 24 for CDRH2;
SEQ ID NO: 11 for CDRH3;
SEQ ID NO: 12 for CDRL1;
SEQ ID NO: 13 for CDRL2; and
SEQ ID NO: 14 for CDRL3.

61. The antigen-binding protein of claim 50 wherein the amino acid sequence of the variable domains of one or both of said single chain Fv is represented by:
SEQ ID NO: 22 for the heavy-chain variable domain (V_{H}); and
SEQ ID NO: 23 for the light-chain variable domain (V_{L}).

62. The antigen-binding protein of claim 50 wherein the nucleotide sequence encoding the variable domains of one or both of said single chain Fv is represented by:
SEQ ID NO: 25 for the heavy-chain variable domain (V_{H}); and
SEQ ID NO: 26 for the light-chain variable domain (V_{L}).

63. The antigen-binding protein of claim 50 wherein one or both of said single chain Fv has a nucleotide sequence represented by SEQ ID NO: 27 or SEQ ID NO: 28.

64. A method for making an antigen-binding protein, which comprises
(a) coexpressing in a host cell
a recombinant DNA construct encoding a first polypeptide having an antigen-binding site located to the N terminus of an immunoglobulin light chain constant domain (C_{L} domain), said C_{L} domain capable of stable association with an immunoglobulin heavy chain first constant domain (C_{H}1 domain), and
a recombinant DNA construct encoding a second polypeptide having an antigen-binding site located to the N terminus of said C_{H}1 domain, said C_{H}1 domain followed by one or more heavy chain constant domains capable of stable self-association,
for a time and in a manner sufficient to allow expression of said polypeptides and formation of said antigen binding protein; and
(b) recovering said antigen binding protein.

65. The method of claim 64 wherein said constructs are on the same DNA expression vector.

66. The method of claim 64 wherein said constructs are on different DNA expression vectors.

67. The method of claim 64 wherein said host cell is a bacterial cell, a yeast cell or a mammalian cell.

68. The method of claim 64 wherein said antigen-binding protein is secreted from the host cell.

69. A method of neutralizing the activation of a VEGF receptor, which comprises treating a cell with an antigen-binding protein of claim 1 in an amount sufficient to neutralize activation of said receptor.

70. The method of claim 69 wherein at least one of the antigen-binding sites is specific for KDR.

71. The method of claim 69 wherein at least one of the antigen-binding sites is specific for FLT1.

72. A method of reducing tumor growth which comprises treating a cell with an antigen-binding protein of claim 1, wherein at least one of the antigen binding sites is specific for a VEGF receptor, in an amount sufficient to reduce tumor growth.

73. The method of claim 72 wherein at least one of the antigen-binding sites is specific for KDR.

74. The method of claim 72 wherein at least one of the antigen-binding sites is specific for FLT1.

75. A method of inhibiting angiogenesis which comprises treating a cell with an antigen-binding protein of claim 1, wherein at least one of the antigen binding sites is specific for a VEGF receptor, in an amount sufficient to inhibit angiogenesis.

76. The method of claim 75 wherein at least one of the antigen-binding sites is specific for KDR.

77. The method of claim 75 wherein at least one of the antigen-binding sites is specific for FLT 1.


SEQUENCE LISTING (34 sequences)

SEQ ID NO: 1 - protein, 10 aa, Mouse
GFNIKDFYMH

SEQ ID NO: 2 - protein, 17 aa, Mouse
WIDPENGDSGYAPKFQG

SEQ ID NO: 3 - protein, 8 aa, Mouse
YYGDYEGY

SEQ ID NO: 4 - protein, 10 aa, Mouse
SASSSVSYMH

SEQ ID NO: 5 - protein, 7 aa, Mouse
STSNLAS

SEQ ID NO: 6 - protein, 9 aa, Mouse
QQRSSYPFT

SEQ ID NO: 7 - protein, 117 aa, Mouse
QVKLQQSGAELVGSGASVKLSCTTSGFNIKDFYMHWVKQRPEQGLEWIGWIDPENGDSGYAPKFQGKATMTADSSSNTAYLQLSSLTSEDTAVYYCNAYYGDYEGYWGQGTTVTVSS

SEQ ID NO: 8 - protein, 108 aa, Mouse
DIELTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIKRA

SEQ ID NO: 9 - DNA, 30 bp, Mouse
ggcttcaacattaaagacttctatatgcac

SEQ ID NO: 10 - DNA, 51 bp, Mouse
tggattgatcctgagaatggtgattctggttatgccccgaagttccagggc

SEQ ID NO: 11 - DNA, 24 bp, Mouse
tactatggtgactacgaaggctac

SEQ ID NO: 12 - DNA, 30 bp, Mouse
agtgccagctcaagtgtaagttacatgcac

SEQ ID NO: 13 - DNA, 21 bp, Mouse
agcacatccaacctggcttct

SEQ ID NO: 14 - DNA, 27 bp, Mouse
cagcaaaggagtagttacccattcacg

SEQ ID NO: 15 - DNA, 351 bp, Mouse
caggtcaagctgcagcagtctggggcagagcttgtggggtcaggggcctcagtcaaattgtcctgcacaacttctggcttcaacattaaagacttctatatgcactgggtgaagcagaggcctgaacagggcctggagtggattggatggattgatcctgagaatggtgattctggttatgccccgaagttccagggcaaggccaccatgactgcagactcatcctccaacacagcctacctgcagctcagcagcctgacatctgaggacactgccgtctattactgtaatgcatactatggtgactacgaaggctactggggccaagggaccacggtcaccgtctcctca

SEQ ID NO: 16 - DNA, 324 bp, Mouse
gacatcgagctcactcagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccattcacgttcggctcggggaccaagctggaaataaaacgggcg

SEQ ID NO: 17 - protein, 15 aa, Mouse
GGGGSGGGGSGGGGS

SEQ ID NO: 18 - DNA, 45 bp, Mouse
ggtggaggcggttcaggcggaggtggctctggcggtggcggatcg

SEQ ID NO: 19 - protein, 10 aa, Mouse
GGGGSGGGGS

SEQ ID NO: 20 - DNA, 15 bp, Mouse
ggtggaggcggttca

SEQ ID NO: 21 - protein, 17 aa, Mouse
WIDPENGDSDYAPKFQG

SEQ ID NO: 22 - protein, 117 aa, Mouse
QVKLQQSGAELVGSGASVKLSCTTSGFNIKDFYMHWVKQRPEQGLEWIGWIDPENGDSDYAPKFQGKATMTADSSSNTAYLQLSSLTSEDTAVYYCNAYYGDYEGYWGQGTTVTVSS

SEQ ID NO: 23 - protein, 106 aa, Mouse
DIELTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIK

SEQ ID NO: 24 - DNA, 51 bp, Mouse
tggattgatcctgagaatggtgattctgattatgccccgaagttccagggc

SEQ ID NO: 25 - DNA, 351 bp, Mouse
caggtcaagctgcagcagtctggggcagagcttgtggggtcaggggcctcagtcaaattgtcctgcacaacttctggcttcaacattaaagacttctatatgcactgggtgaagcagaggcctgaacagggcctggagtggattggatggattgatcctgagaatggtgattctgattatgccccgaagttccagggcaaggccaccatgactgcagactcatcctccaacacagcctacctgcagctcagcagcctgacatctgaggacactgccgtctattactgtaatgcatactatggtgactacgaaggctactggggccaagggaccacggtcaccgtctcctca

SEQ ID NO: 26 - DNA, 318 bp, Mouse
gacatcgagctcactcagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccattcacgttcggctcggggaccaagctggaaataaaa

SEQ ID NO: 27 - protein, 240 aa, Mouse
QVKLQQSGAELVGSGASVKLSCTTSGFNIKDFYMHWVKQRPEQGLEWIGWIDPENGDSGYAPKFQGKATMTADSSSNTAYLQLSSLTSEDTAVYYCNAYYGDYEGYWGQGTTVTVSSGGGGSGGGGSGGGGSDIELTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIKRA

SEQ ID NO: 28 - protein, 238 aa, Mouse
QVKLQQSGAELVGSGASVKLSCTTSGFNIKDFYMHWVKQRPEQGLEWIGWIDPENGDSDYAPKFQGKATMTADSSSNTAYLQLSSLTSEDTAVYYCNAYYGDYEGYWGQGTTVTVSSGGGGSGGGGSGGGGSDIELTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIK

SEQ ID NO: 29 - DNA, 43 bp, Artificial Sequence
  Synthetic primer
ctagtagcaactgccaccggcgtacattcacaggtcaagctgc

SEQ ID NO: 30 - DNA, 30 bp, Artificial Sequence
  Synthetic primer
tcgaaggatcactcaccttttatttccagc

SEQ ID NO: 31 - DNA, 52 bp, Artificial Sequence
  Synthetic primer
ggtcaaaagcttatggggatggtcatgtatcatcctttttctagtagcaact

SEQ ID NO: 32 - DNA, 36 bp, Artificial Sequence
  Signal
tcgatctagaaggatccactcacgttttatttccag

SEQ ID NO: 33 - protein, 19 aa, Artificial Sequence
  leader peptide
MGWSCIILFLVATATGVHS

SEQ ID NO: 34 - DNA, 32 bp, Artificial Sequence
  Synthetic primer
tctcggccggcttaagctgcgcatgtgtgagt